(19) United States
(12) Patent Application Publication
(10) Pub. No.: US 2026/0252805 A1
(43) Pub. Date: Aug. 27, 2026

(54) SYSTEM

(71) Applicant: SoftBank Group Corp., Tokyo (JP)

(72) Inventor: Taito KAWASE, Tokyo (JP)

(73) Assignee: SoftBank Group Corp., Tokyo (JP)

(21) Appl. No.: 19/536,267

(22) Filed: Feb. 11, 2026

(30) Foreign Application Priority Data
Feb. 21, 2025 (JP) 2025-027034

(51) Int. Cl. G06F 40/30

(52) U.S. Cl. CPC G06F 40/30

(57) ABSTRACT

The system according to the embodiment comprises an imaging unit, an analysis unit, an instruction unit, and a confirmation unit. The imaging unit captures images of the inside of the mouth. The analysis unit analyzes images captured by the imaging unit and determines the presence of tartar or unbrushed areas. The instruction unit informs the user where to brush based on the result determined by the analysis unit. The confirmation unit captures the inside of the mouth again and performs confirmation after the user has brushed their teeth according to the instructions.

### CROSS-REFERENCE TO RELATED APPLICATION

The present application claims priority to and incorporates by reference the entire contents of Japanese Patent Application No. 2025-027034 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, it has been difficult to easily perform dental care at home, resulting in the problem of requiring the effort to visit a dentist.

### SUMMARY OF THE INVENTION

The system according to the embodiment comprises an imaging unit, an analysis unit, an instruction unit, and a confirmation unit. The imaging unit captures images of the inside of the mouth. The analysis unit analyzes images captured by the imaging unit and determines the presence of tartar or unbrushed areas. The instruction unit informs the user where to brush based on the result determined by the analysis unit. The confirmation unit captures the inside of the mouth again and performs confirmation after the user has brushed their teeth according to the instructions.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

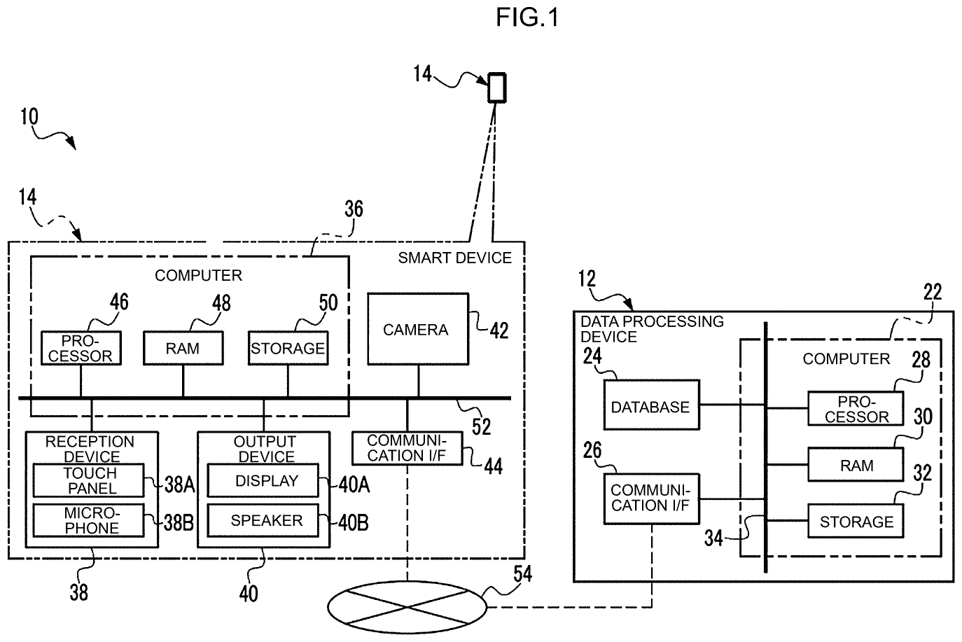

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

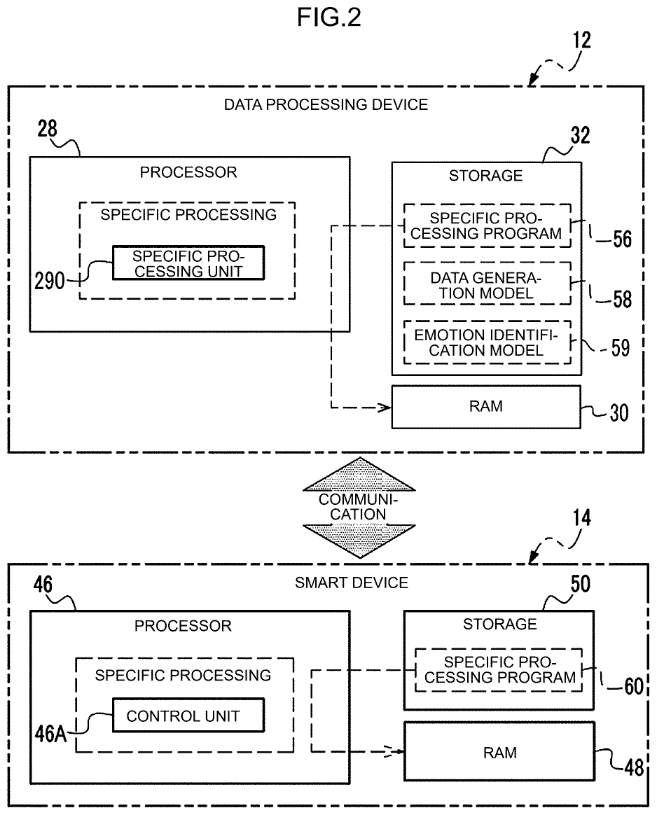

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The dental care system according to the embodiment of the present invention is a system that enables easy dental care at home. This dental care system allows a user to capture images of the inside of the mouth with a camera, and AI analyzes the images to determine the presence of tartar or unbrushed areas, and instructs the user where to brush. After the user brushes their teeth according to the instructions, the inside of the mouth is captured again with the camera, and AI performs confirmation again. With this system, the user can easily perform dental care at home and eliminate the need to visit a dentist. For example, the part where the user captures images of the inside of the mouth with a camera is referred to as the “imaging unit,” and the part where AI analyzes the images is referred to as the “analysis unit.” The analysis unit analyzes the images to determine the presence of tartar or unbrushed areas. Next, based on the result of the analysis unit, the part that instructs the user where to brush is referred to as the “instruction unit.” Finally, after the user brushes their teeth according to the instructions, the part where the inside of the mouth is captured again with the camera and AI performs confirmation again is referred to as the “confirmation unit.” Thus, the dental care system enables the user to easily perform dental care at home and eliminates the need to visit a dentist. Specifically, the dental care system is composed of multiple hardware and software modules, namely the imaging unit, analysis unit, instruction unit, and confirmation unit. The imaging unit has a function to acquire images of the oral cavity using an imaging device such as a smartphone or dedicated camera, and the image data is stored as an RGB three-dimensional tensor (e.g., 224×224×3 pixels). The imaging unit is equipped with features such as switching between wide-angle and macro lenses, lighting control, and autofocus, and can automatically set optimal image acquisition conditions according to user operation. The analysis unit uses image recognition models such as convolutional neural networks (CNN) or Vision Transformer, takes the image tensor obtained from the imaging unit as input, and determines the presence and location of tartar or unbrushed areas. Examples of input include overall oral images, enlarged images of specific areas, and images under different lighting conditions. The output of the analysis unit is structured data such as binary masks of tartar regions (224×224 binary images), probability maps of unbrushed areas (continuous values from 0 to 1), and labels for each area (e.g., upper left molar, lower right incisor, etc.). The analysis unit can continuously improve determination accuracy through transfer learning using pre-trained models and continual learning using user-specific history data. The instruction unit, based on the output of the analysis unit, highlights unbrushed areas in red on the user interface or generates specific instructions such as “Please brush the upper right molar a little more” using a speech synthesis engine. The instruction unit can also personalize instruction content and expression methods by considering the user's past brushing habits and emotional state (e.g., tension, relaxation). Furthermore, the confirmation unit, after the user brushes their teeth according to the instructions, inputs the newly acquired image from the imaging unit into the analysis unit for re-determination of unbrushed areas. The confirmation unit performs actions such as notifying the timing for re-imaging, re-highlighting unbrushed areas, and providing feedback on improvement points (e.g., brushing guide videos). These series of processes are executed rapidly on parallel computing clusters using GPUs or cloud servers, allowing the user to receive real-time feedback. As a technical effect, this system, unlike conventional care relying on human visual confirmation and empirical rules, achieves highly accurate determination and personalized instruction generation using image recognition AI, greatly improving the detection accuracy of tartar and unbrushed areas and care efficiency. In addition, by integrally analyzing multidimensional data such as the user's brushing habits, emotional state, and health history, an optimized care cycle for each individual can be automated. Application fields include self-care support in general households, remote monitoring for nursing facilities and the elderly, pre-screening in dental clinics, and tooth brushing instruction in educational settings.

The dental care system according to the embodiment comprises an imaging unit, an analysis unit, an instruction unit, and a confirmation unit. The imaging unit allows the user to capture images of the inside of the mouth with a camera. When capturing images of the inside of the mouth, for example, a smartphone camera or a dedicated digital camera may be used. The imaging unit may use a wide-angle lens to capture the entire inside of the mouth. The imaging unit may also use a macro lens to capture detailed images of specific areas inside the mouth. The analysis unit uses AI to analyze images captured by the imaging unit and determines the presence of tartar or unbrushed areas. The analysis unit, for example, can use deep learning technology to detect tartar or unbrushed areas in images with high accuracy. The analysis unit may also use neural networks to identify the location of tartar or unbrushed areas in images. The instruction unit, based on the result determined by the analysis unit, instructs the user where to brush. The instruction unit, for example, can visually indicate where to brush by highlighting unbrushed areas for the user. The instruction unit may also use voice guidance to instruct the user on how to brush. The confirmation unit, after the user brushes their teeth according to the instructions, captures images of the inside of the mouth again with the camera, and AI performs confirmation again. The confirmation unit, for example, can notify the user of the timing for re-imaging and analyze the newly captured images to confirm whether there are any unbrushed areas. Thus, the dental care system according to the embodiment enables the user to easily perform dental care at home and eliminates the need to visit a dentist. Specifically, the dental care system is composed of multiple hardware and software modules, namely the imaging unit, analysis unit, instruction unit, and confirmation unit. The imaging unit has a function to acquire images of the oral cavity using an imaging device such as a smartphone or dedicated camera, and the image data is stored as an RGB three-dimensional tensor (e.g., 224×224×3 pixels). The imaging unit is equipped with features such as switching between wide-angle and macro lenses, lighting control, and autofocus, and can automatically set optimal image acquisition conditions according to user operation. The analysis unit uses image recognition models such as convolutional neural networks (CNN) or Vision Transformer, takes the image tensor obtained from the imaging unit as input, and determines the presence and location of tartar or unbrushed areas. Examples of input include overall oral images, enlarged images of specific areas, and images under different lighting conditions. The output of the analysis unit is structured data such as binary masks of tartar regions (224×224 binary images), probability maps of unbrushed areas (continuous values from 0 to 1), and labels for each area (e.g., upper left molar, lower right incisor, etc.). The analysis unit can continuously improve determination accuracy through transfer learning using pre-trained models and continual learning using user-specific history data. The instruction unit, based on the output of the analysis unit, highlights unbrushed areas in red on the user interface or generates specific instructions such as “Please brush the upper right molar a little more” using a speech synthesis engine. The instruction unit can also personalize instruction content and expression methods by considering the user's past brushing habits and emotional state (e.g., tension, relaxation). Furthermore, the confirmation unit, after the user brushes their teeth according to the instructions, inputs the newly acquired image from the imaging unit into the analysis unit for re-determination of unbrushed areas. The confirmation unit performs actions such as notifying the timing for re-imaging, re-highlighting unbrushed areas, and providing feedback on improvement points (e.g., brushing guide videos). These series of processes are executed rapidly on parallel computing clusters using GPUs or cloud servers, allowing the user to receive real-time feedback. As a technical effect, this system, unlike conventional care relying on human visual confirmation and empirical rules, achieves highly accurate determination and personalized instruction generation using image recognition AI, greatly improving the detection accuracy of tartar and unbrushed areas and care efficiency. In addition, by integrally analyzing multidimensional data such as the user's brushing habits, emotional state, and health history, an optimized care cycle for each individual can be automated. Application fields include self-care support in general households, remote monitoring for nursing facilities and the elderly, pre-screening in dental clinics, and tooth brushing instruction in educational settings.

The analysis unit can analyze images using AI and determine the presence of tartar or unbrushed areas. For example, the analysis unit can use deep learning technology to detect tartar or unbrushed areas in captured images with high accuracy. Deep learning technology can automatically extract features from a large amount of image data and determine the presence of tartar or unbrushed areas. The analysis unit may also use neural networks to identify the location of tartar or unbrushed areas in images. Neural networks are artificial intelligence models consisting of multiple layers, which process input image data layer by layer and ultimately output the location of tartar or unbrushed areas. Furthermore, the analysis unit can use image processing algorithms to highlight tartar or unbrushed areas in images. For example, by highlighting tartar or unbrushed areas in red in the image, the user can easily visually confirm them. Thus, by using AI, the analysis unit improves the accuracy of determining tartar or unbrushed areas. Some or all of the above-described processing in the analysis unit may be performed using AI or without using AI. For example, the analysis unit may input captured image data to a generative AI and have the generative AI determine tartar or unbrushed areas from the image data. Specifically, the analysis unit receives oral cavity images obtained from the imaging unit as RGB three-dimensional tensors (e.g., 224×224×3 pixels), and performs preprocessing such as contrast adjustment, noise removal, and normalization (scaling to mean 0, standard deviation 1). The analysis unit uses image recognition models such as convolutional neural networks (CNN) or Vision Transformer, extracts features from the input tensor through multiple convolutional layers, pooling layers, and fully connected layers, and determines the presence of tartar or unbrushed areas. For example, in the case of CNN, the first layer extracts edge and texture features, the intermediate layers detect tooth contours and tartar shape patterns, and the final layer outputs area labels (e.g., upper left molar, lower right incisor), binary masks of tartar regions (224×224 binary images), and probability maps of unbrushed areas (continuous values from 0 to 1). Examples of input include overall oral images, enlarged images of specific areas, and images under different lighting conditions. The analysis unit continuously improves determination accuracy through transfer learning using pre-trained models and continual learning (online learning) using user-specific history data. Based on the output binary masks and probability maps, the analysis unit applies image processing algorithms (e.g., morphology processing, contour extraction, heatmap generation) to highlight tartar or unbrushed areas in red or with semi-transparent overlays. The analysis unit sends these outputs as structured data to the instruction unit and confirmation unit for subsequent user interface display and voice guide generation. Examples of input to AI include “224×224×3 oral cavity image tensor,” “128×128×3 enlarged image of lower right molar,” and “multiple image tensors captured under different lighting conditions.” Examples of output from AI include “tartar region binary mask (224×224),” “unbrushed area probability map (224×224, value range 0-1),” and “area labels (e.g., upper left molar: tartar present, lower right incisor: no unbrushed area).” The analysis unit uses these outputs for threshold determination and rule-based processing as branching conditions for warnings or instruction generation to the user. As a technical effect, the analysis unit, without relying on human visual inspection or empirical rules, greatly improves the detection accuracy and reproducibility of tartar and unbrushed areas through high-dimensional feature extraction and pattern recognition by image recognition AI. Furthermore, high-speed inference using parallel computing clusters with GPUs or cloud servers enables real-time determination and feedback. Application fields include self-care support in general households, remote monitoring for nursing facilities and the elderly, pre-screening in dental clinics, and tooth brushing instruction in educational settings.

The instruction unit can provide instructions to the user based on the result of the analysis unit. For example, the instruction unit can visually indicate to the user the areas of unbrushed teeth determined by the analysis unit. The instruction unit can highlight unbrushed areas in red, allowing the user to visually confirm where to brush. The instruction unit may also use voice guidance to instruct the user on how to brush. For example, the instruction unit can provide specific instructions to the user by voice, such as “Please brush the upper right molar a little more.” Furthermore, the instruction unit can analyze the user's brushing habits and present specific improvement points. For example, the instruction unit can analyze the user's brushing data, identify areas with frequent unbrushed spots or brushing patterns, and present improvement points based on that analysis. Thus, the instruction unit can provide appropriate instructions to the user based on the analysis results. Some or all of the above-described processing in the instruction unit may be performed using AI or without using AI. For example, the instruction unit may input the result determined by the analysis unit to a generative AI and have the generative AI generate instructions for the user. Specifically, the instruction unit receives structured data from the analysis unit (e.g., tartar region binary mask, unbrushed area probability map, area labels, etc.) as input and performs image generation processing to highlight unbrushed areas in red or with semi-transparent overlays on the user interface. The instruction unit incorporates a speech synthesis engine and can automatically generate specific instruction sentences such as “Please brush the outside of the upper right molar a little more” or “There are unbrushed areas on the back side of the lower left incisor” based on the output of the analysis unit, and output them as voice data. The instruction unit accumulates the user's past brushing data (e.g., heatmap of unbrushed area frequency, time-series patterns of brushing, brushing pressure sensor values, etc.) as time-series vectors, and uses recurrent neural networks (RNN) or autoregressive models to extract brushing habits and tendencies. Based on the extracted features, the instruction unit generates personalized improvement points for each user (e.g., “There are many unbrushed areas on the lower left molar, so try changing the angle of the brush,” etc.). Examples of input to AI include “224×224 unbrushed area probability map,” “array of unbrushed area labels for the past 30 times,” and “time-series data of brushing pressure.” Examples of output from AI include “highlighted images of unbrushed areas,” “voice instruction text,” and “list of improvement points (e.g., lower left molar: adjust brush angle, upper right incisor: extend brushing time).” The instruction unit sends these outputs to the user interface or voice output module for real-time feedback to the user. The instruction unit utilizes generative AI (e.g., large language models or multimodal generative models) to convert structured data from the analysis unit into natural language instructions or image/voice guides, thereby achieving optimized instruction expressions for each user. As a technical effect, the instruction unit, unlike conventional static instruction displays or simple warning sounds, can greatly improve the user's rate of improvement in unbrushed areas and care continuity through high-dimensional feature extraction and personalized instruction generation by AI. Furthermore, by integrally analyzing multidimensional data such as the user's brushing history and emotional state, the instruction content and expression methods can be dynamically optimized to enhance the quality of the user experience. Application fields include self-care support in general households, remote guidance for nursing facilities and the elderly, patient education in dental clinics, and tooth brushing instruction in educational settings.

The confirmation unit can capture images of the inside of the mouth with a camera and perform confirmation using AI after the user has brushed their teeth according to the instructions. For example, the confirmation unit can notify the user to capture images of the inside of the mouth again after brushing according to the instructions. The confirmation unit analyzes the newly captured images to confirm whether there are any unbrushed areas. For example, the confirmation unit can notify the user of the timing for re-imaging and analyze the newly captured images to confirm whether there are any unbrushed areas. The confirmation unit can highlight unbrushed areas in the newly captured images, making it easier for the user to visually confirm them. The confirmation unit can also accumulate the user's brushing data and analyze long-term brushing trends. For example, the confirmation unit can analyze the user's brushing data over time and identify patterns of unbrushed areas. Thus, by confirming the state after brushing, the confirmation unit can prevent unbrushed areas. Some or all of the above-described processing in the confirmation unit may be performed using AI or without using AI. For example, the confirmation unit may input the newly captured image data to a generative AI and have the generative AI confirm unbrushed areas from the image data. Specifically, after the user has brushed their teeth according to instructions from the instruction unit, the confirmation unit receives the latest oral cavity image obtained by the imaging unit as an RGB three-dimensional tensor (e.g., 224×224×3 pixels). Immediately after image acquisition, the confirmation unit sends a push notification to the user for the timing of re-imaging, and after the user completes imaging, the image data is preprocessed (contrast adjustment, noise removal, normalization) and sent to the analysis unit. The confirmation unit receives structured data such as binary masks of tartar regions and probability maps of unbrushed areas returned from the analysis unit, and performs image generation processing to highlight unbrushed areas in red or with semi-transparent overlays. Furthermore, the confirmation unit accumulates the user's brushing history data (e.g., array of unbrushed area labels for the past 30 times, time-series vector of brushing pressure sensor values, etc.) in a time-series database, and uses recurrent neural networks (RNN) or autoregressive models to extract long-term trends of unbrushed areas and improvement patterns. Based on the extracted trends, the confirmation unit identifies areas with frequent unbrushed spots or brushing habits, and utilizes this information for feedback to the instruction unit for future instructions or for presenting improvement points to the user. Examples of input to AI include “224×224×3 re-imaged oral cavity image tensor,” “array of unbrushed area labels for the past 30 times,” and “time-series data of brushing pressure.” Examples of output from AI include “binary mask of unbrushed areas (224×224),” “probability map of unbrushed areas (224×224, value range 0-1),” and “list of improvement points (e.g., lower left molar: adjust brush angle, upper right incisor: extend brushing time).” The confirmation unit uses these outputs for threshold determination and rule-based processing as branching conditions for warnings or instruction generation to the user. In subsequent processing, if the amount of unbrushed areas exceeds a certain threshold, further brushing instructions are issued; if below the threshold, care completion notification or recommended timing for next care is presented. As a technical effect, the confirmation unit, unlike conventional human visual confirmation or empirical rules, greatly improves the detection accuracy, reproducibility, and optimization of the care cycle for unbrushed areas through high-dimensional feature extraction, pattern recognition, and time-series analysis by image recognition AI. Furthermore, by integrally analyzing the user's brushing habits and history data, the care cycle can be optimized for each individual, enhancing the quality of the user experience. Application fields include self-care support in general households, remote monitoring for nursing facilities and the elderly, pre-screening in dental clinics, and tooth brushing instruction in educational settings.

The imaging unit can estimate the user's emotion and adjust the imaging timing based on the estimated emotion of the user. For example, the imaging unit can capture the user's facial expression with a camera and estimate the user's emotion using an emotion estimation algorithm. For example, the imaging unit can calculate an emotion score based on changes in the user's facial expression and determine whether the user is relaxed or tense. The imaging unit may also record the user's voice and estimate the user's emotion using voice analysis technology. For example, the imaging unit can analyze the tone and speed of the user's voice and calculate an emotion score. Furthermore, the imaging unit can collect the user's biometric data (heart rate and skin conductance) with sensors and estimate emotion using an emotion estimation algorithm. For example, the imaging unit can calculate an emotion score based on fluctuations in the user's heart rate. Thus, the imaging unit can adjust the imaging timing according to the user's emotion. Emotion estimation may be realized using an emotion engine or generative AI, such as a text generation AI (e.g., LLM) or multimodal generative AI, but is not limited to these examples. Some or all of the above-described processing in the imaging unit may be performed using AI or without using AI. For example, the imaging unit may input the user's facial expression data to a generative AI and have the generative AI estimate emotion. Specifically, the imaging unit simultaneously acquires the user's facial image (e.g., 128×128×3 pixel RGB tensor), voice waveform data (e.g., 1-second time-series array sampled at 16 kHz), and biometric sensor data such as heart rate time-series (e.g., 60-element vector sampled at 1 Hz) and skin conductance values (e.g., 60-element vector sampled at 1 Hz), and inputs these to an emotion estimation AI model. The imaging unit applies a convolutional neural network (CNN) to the facial image to extract facial features (e.g., degree of mouth corner lift, frown lines, eye opening, etc.). For voice data, it extracts acoustic features such as Mel-frequency cepstral coefficients (MFCC), pitch, and formants, and inputs them to a recurrent neural network (RNN) or Transformer-based voice emotion recognition model. For biometric data, it extracts features such as heart rate variability and skin conductance fluctuation patterns and inputs them to a multilayer perceptron (MLP) or time-series model. The AI model integrates these multimodal features and outputs emotion classes (e.g., relaxed, tense, stressed, joyful) and emotion scores (e.g., continuous values from 0 to 1). Examples of input include “128×128×3 facial image tensor,” “1-second voice waveform array,” “60-element heart rate vector,” and “60-element skin conductance vector.” Examples of output from AI include “emotion class: relaxed, emotion score: 0.85,” “emotion class: tense, emotion score: 0.30,” etc. The imaging unit automatically adjusts the imaging timing only when the output emotion score exceeds a certain threshold (e.g., 0.7 or higher for relaxed, 0.3 or lower for tense), executing imaging when the user is relaxed or temporarily delaying imaging when the user is tense. In subsequent processing, the emotion estimation result is sent to the analysis unit or instruction unit and used for interaction adjustment to optimize the user experience and reduce stress. As a technical effect, the imaging unit can estimate the user's emotional state with high accuracy from multimodal data and perform imaging at the optimal timing, thereby reducing user stress and discomfort and enabling image acquisition with natural expressions and states. This also improves the determination accuracy in the analysis unit and the quality of personalized instruction generation in the instruction unit. Application fields include self-care support in general households, stress-free remote monitoring for nursing facilities and the elderly, reduction of patient burden in dental clinics, and tooth brushing instruction for children in educational settings. Furthermore, the emotion estimation AI model is optimized for each user through transfer learning and continual learning, improving accuracy over long-term care cycles.

The imaging unit can display a guide for focusing on specific areas inside the mouth during imaging. For example, when the user points the camera at the inside of the mouth, the imaging unit can display guidelines indicating specific areas. For example, when the user moves the camera, the imaging unit can highlight the areas that should be focused on. The imaging unit can also continue to display guidelines until the user captures the specific area. For example, when the user moves the camera, the imaging unit can display arrows or frames indicating specific areas and continue to display the guidelines until the user captures those areas. Thus, by focusing on specific areas during imaging, the imaging unit can acquire detailed images. Some or all of the above-described processing in the imaging unit may be performed using AI or without using AI. For example, the imaging unit may input the user's camera operation data to a generative AI and have the generative AI instruct which areas should be focused on. Specifically, when the user captures images of the oral cavity using a smartphone or dedicated camera, the imaging unit acquires real-time camera orientation and position information (e.g., gyro sensor values, accelerometer values, camera field of view information, etc.) and records these camera operation data as time-series vectors (e.g., 30 samples per second of 6-axis sensor data). The imaging unit overlays guidelines (e.g., semi-transparent frames, arrows, blinking markers, etc.) on the user interface based on coordinate information of unbrushed areas or tartar detection regions received from the analysis unit (e.g., bounding box coordinates or segmentation masks in the image). Each time the user moves the camera, the imaging unit uses image recognition AI (e.g., object detection CNN or pose estimation model) to determine in real time whether the current camera view sufficiently captures the target area. Examples of input to AI include “current camera image frame (224×224×3 tensor),” “time-series vector of camera orientation and position,” and “list of coordinates of unbrushed areas from the analysis unit.” Examples of output from AI include “guide display position (image coordinates: x=120, y=80, width=40, height=40),” “label of area to be highlighted (e.g., lower right molar),” etc. The imaging unit continues to display the guidelines until the user sufficiently captures the specified area, and removes the guide only when imaging is determined to be complete. Furthermore, the imaging unit can learn the user's past imaging history and camera operation habits (e.g., tendency to have difficulty imaging specific areas) and personalize the guide display method and timing for future imaging. As a technical effect, the imaging unit, unlike conventional simple overall imaging or user-dependent imaging, can greatly improve the acquisition rate of high-resolution images of key areas through real-time area guidance and guide display by AI. This improves the detection accuracy of tartar and unbrushed areas in the analysis unit and enhances the overall care quality of the system. Application fields include self-care support in general households, remote monitoring in nursing facilities, pre-screening in dental clinics, and tooth brushing instruction in educational settings.

The imaging unit can measure humidity and temperature inside the mouth during imaging and automatically adjust imaging conditions. For example, before imaging, the imaging unit can measure the humidity inside the mouth and set optimal imaging conditions. For example, the imaging unit can use a humidity sensor to measure the humidity inside the mouth and adjust the camera's exposure or white balance according to the humidity. The imaging unit can also measure the temperature inside the mouth during imaging and adjust imaging conditions as needed. For example, the imaging unit can use a temperature sensor to measure the temperature inside the mouth and automatically adjust the camera settings according to the temperature. Furthermore, after imaging, the imaging unit can record the humidity and temperature inside the mouth and use them for future imaging. For example, the imaging unit can save the humidity and temperature data at the time of imaging and refer to them for the next imaging. Thus, by setting optimal imaging conditions according to the humidity and temperature inside the mouth, the imaging unit can acquire higher-quality images. Some or all of the above-described processing in the imaging unit may be performed using AI or without using AI. For example, the imaging unit may input humidity and temperature data to a generative AI and have the generative AI set optimal imaging conditions. Specifically, the imaging unit acquires real-time data from humidity sensors (e.g., digital humidity sensor, measurement range 0-100% RH, resolution 0.1% RH) and temperature sensors (e.g., thermistor, measurement range 20-45° C., resolution 0.1° C.) placed inside the oral cavity. The imaging unit records these sensor values as time-series vectors (e.g., 10 samples per second of humidity and temperature data) and sends them to the camera control module before, during, and after imaging. If the humidity is high, the imaging unit shortens the camera's exposure time and applies a lens fog correction algorithm (e.g., image contrast enhancement, histogram equalization). If the temperature is high, the imaging unit automatically adjusts the white balance and applies a color temperature correction filter. Examples of input to AI include “current humidity value: 85% RH,” “current temperature value: 37.2° C.,” and “history vector of humidity and temperature for the past 10 times.” Examples of output from AI include “exposure time setting: 1/100 sec,” “white balance setting: color temperature 5500K,” and “fog correction algorithm application flag.” After imaging, the imaging unit saves humidity and temperature data to a user-specific history database and can preset optimal imaging parameters for the next imaging by referring to past environmental conditions. Furthermore, the AI model learns the correlation between past image quality and environmental conditions and automates the optimization of image sharpness and color information. As a technical effect, the imaging unit, unlike conventional fixed imaging conditions or user-dependent settings, achieves consistently high-quality image acquisition under varying humidity and temperature conditions through real-time environmental sensing and dynamic parameter optimization by AI. This improves the detection accuracy of tartar and unbrushed areas in the analysis unit and enhances the reliability of the entire system. Application fields include self-care support in general households, remote monitoring in nursing facilities, pre-screening in dental clinics, and tooth brushing instruction in educational settings.

The imaging unit can estimate the user's emotion and adjust the imaging frequency based on the estimated emotion of the user. For example, the imaging unit can capture the user's facial expression with a camera and estimate the user's emotion using an emotion estimation algorithm. For example, the imaging unit can calculate an emotion score based on changes in the user's facial expression and determine whether the user is relaxed or tense. The imaging unit may also record the user's voice and estimate the user's emotion using voice analysis technology. For example, the imaging unit can analyze the tone and speed of the user's voice and calculate an emotion score. Furthermore, the imaging unit can collect the user's biometric data (heart rate and skin conductance) with sensors and estimate emotion using an emotion estimation algorithm. For example, the imaging unit can calculate an emotion score based on fluctuations in the user's heart rate. Thus, the imaging unit can adjust the imaging frequency according to the user's emotion. Emotion estimation may be realized using an emotion engine or generative AI, such as a text generation AI (e.g., LLM) or multimodal generative AI, but is not limited to these examples. Some or all of the above-described processing in the imaging unit may be performed using AI or without using AI. For example, the imaging unit may input the user's facial expression data to a generative AI and have the generative AI estimate emotion. Specifically, the imaging unit simultaneously acquires the user's facial image (e.g., 128×128×3 pixel RGB tensor), voice waveform data (e.g., 1-second time-series array sampled at 16 kHz), and biometric sensor data such as heart rate time-series (e.g., 60-element vector sampled at 1 Hz) and skin conductance values (e.g., 60-element vector sampled at 1 Hz), and inputs these to an emotion estimation AI model. The imaging unit applies a convolutional neural network (CNN) to the facial image to extract facial features (e.g., degree of mouth corner lift, frown lines, eye opening, etc.). For voice data, it extracts acoustic features such as Mel-frequency cepstral coefficients (MFCC), pitch, and formants, and inputs them to a recurrent neural network (RNN) or Transformer-based voice emotion recognition model. For biometric data, it extracts features such as heart rate variability and skin conductance fluctuation patterns and inputs them to a multilayer perceptron (MLP) or time-series model. The AI model integrates these multimodal features and outputs emotion classes (e.g., relaxed, tense, stressed, joyful) and emotion scores (e.g., continuous values from 0 to 1). Examples of input include “128×128×3 facial image tensor,” “1-second voice waveform array,” “60-element heart rate vector,” and “60-element skin conductance vector.” Examples of output from AI include “emotion class: relaxed, emotion score: 0.85,” “emotion class: tense, emotion score: 0.30,” etc. The imaging unit automatically adjusts the imaging frequency only when the output emotion score exceeds a certain threshold (e.g., 0.7 or higher for relaxed, 0.3 or lower for tense), increasing the imaging frequency when the user is relaxed and decreasing or temporarily delaying the imaging frequency when the user is tense. In subsequent processing, the emotion estimation result is sent to the analysis unit or instruction unit and used for interaction adjustment to optimize the user experience and reduce stress. As a technical effect, the imaging unit can estimate the user's emotional state with high accuracy from multimodal data and perform imaging at the optimal frequency, thereby reducing user stress and discomfort and enabling image acquisition with natural expressions and states. This also improves the determination accuracy in the analysis unit and the quality of personalized instruction generation in the instruction unit. Application fields include self-care support in general households, stress-free remote monitoring for nursing facilities and the elderly, reduction of patient burden in dental clinics, and tooth brushing instruction for children in educational settings. Furthermore, the emotion estimation AI model is optimized for each user through transfer learning and continual learning, improving accuracy over long-term care cycles.

The imaging unit can select an imaging mode according to the health condition of the user's oral cavity during imaging. For example, if the user's oral cavity is healthy, the imaging unit can select a normal imaging mode. For example, if there are no abnormalities in the user's oral cavity, the imaging unit can use a standard imaging mode. If there are abnormalities in the user's oral cavity, the imaging unit can select a detailed imaging mode. For example, if there are abnormalities in the user's oral cavity, the imaging unit can use a high-resolution imaging mode to acquire detailed images. Furthermore, if the user's oral cavity is dry, the imaging unit can select an imaging mode that considers humidity. For example, if the user's oral cavity is dry, the imaging unit can use a humidity sensor to measure humidity and select an imaging mode according to the humidity. Thus, by selecting an imaging mode according to the health condition of the user's oral cavity, the imaging unit can acquire appropriate images. Some or all of the above-described processing in the imaging unit may be performed using AI or without using AI. For example, the imaging unit may input the user's oral health condition data to a generative AI and have the generative AI select the optimal imaging mode. Specifically, the imaging unit receives oral health condition data from the analysis unit (e.g., presence or absence of tartar detection, unbrushed area probability map, dryness score, etc.) and humidity/temperature sensor values (e.g., humidity 65% RH, temperature 36.5° C.) as input. If the health condition is determined to be “no abnormality,” the imaging unit automatically selects a standard resolution (e.g., 224×224 pixels), standard exposure, and standard white balance imaging mode. If the health condition is determined to be “abnormal” (e.g., tartar detected, signs of inflammation, high dryness), the imaging unit automatically selects a high-resolution (e.g., 512×512 pixels), macro lens switching, extended exposure time, and special lighting (e.g., blue LED for fluorescence observation) detailed imaging mode. If the humidity is low, the imaging unit applies a lens fog prevention algorithm or contrast enhancement filter. Examples of input to AI include “health condition label: no abnormality,” “humidity value: 65% RH,” and “dryness score: 0.8.” Examples of output from AI include “imaging mode: standard,” “imaging mode: high resolution/macro,” and “special lighting: ON.” The imaging unit can learn the user's health condition history and past image quality data to personalize imaging mode selection for future imaging. As a technical effect, the imaging unit, unlike conventional uniform imaging modes, achieves highly accurate image acquisition even for abnormal areas or dry conditions through dynamic imaging mode optimization based on AI health condition determination and environmental sensing. This improves the accuracy of abnormality detection in the analysis unit and the degree of personalization of care instructions, enhancing the reliability of the entire system. Application fields include self-care support in general households, remote monitoring in nursing facilities, pre-screening in dental clinics, and tooth brushing instruction in educational settings.

The imaging unit can detect movement inside the user's oral cavity during imaging and add a function to correct the movement. For example, when the user moves their mouth, the imaging unit can automatically correct blurring. For example, when the user moves their mouth, the imaging unit can detect the movement and automatically adjust the camera settings to minimize blurring. The imaging unit can also minimize blurring when the user moves the camera. For example, when the user moves the camera, the imaging unit can detect the movement and automatically adjust the camera settings to correct blurring. Furthermore, if the user moves during imaging, the imaging unit can correct blurring in real time. For example, when the user moves during imaging, the imaging unit can detect the movement and adjust the camera settings in real time to correct blurring. Thus, by detecting movement inside the user's oral cavity and correcting blurring, the imaging unit can acquire clearer images. Some or all of the above-described processing in the imaging unit may be performed using AI or without using AI. For example, the imaging unit may input the user's movement data to a generative AI and have the generative AI perform blurring correction. Specifically, the imaging unit acquires real-time movement data (e.g., XYZ axis acceleration, angular velocity, sampling rate 100 Hz) from an accelerometer or gyro sensor built into the camera body and records it as a time-series vector. The imaging unit applies image recognition AI (e.g., optical flow estimation CNN, movement correction RNN) to image frames during imaging (e.g., 224×224×3 pixels) to estimate movement vectors and blurring amounts between frames. Examples of input to AI include “image tensors of two consecutive frames,” “100 samples of acceleration and angular velocity vectors,” etc. Examples of output from AI include “estimated blurring amount: 3.2 pixels,” “corrected image frame,” and “camera setting change instructions (e.g., shutter speed 1/500 sec, image stabilization ON).” The imaging unit automatically adjusts the camera's shutter speed, ISO sensitivity, and image stabilization mechanism (e.g., electronic image stabilization, image stabilizer) in real time according to the estimated movement and blurring amount. Furthermore, the AI model can learn the user's movement tendencies and past blurring occurrence history to personalize correction parameters for future imaging. As a technical effect, the imaging unit, unlike conventional static imaging or simple image stabilization, achieves consistently clear image acquisition that is robust to user and camera movement through multimodal movement detection and real-time correction by AI. This improves the detection accuracy of tartar and unbrushed areas in the analysis unit and enhances the reliability of the entire system. Application fields include self-care support in general households, remote monitoring in nursing facilities, pre-screening in dental clinics, and tooth brushing instruction in educational settings.

The analysis unit can estimate the user's emotion and adjust the display method of the analysis result based on the estimated emotion of the user. For example, the analysis unit can capture the user's facial expression with a camera and estimate the user's emotion using an emotion estimation algorithm. For example, the analysis unit can calculate an emotion score based on changes in the user's facial expression and determine whether the user is relaxed or tense. The analysis unit may also record the user's voice and estimate the user's emotion using voice analysis technology. For example, the analysis unit can analyze the tone and speed of the user's voice and calculate an emotion score. Furthermore, the analysis unit can collect the user's biometric data (heart rate and skin conductance) with sensors and estimate emotion using an emotion estimation algorithm. For example, the analysis unit can calculate an emotion score based on fluctuations in the user's heart rate. Thus, the analysis unit can adjust the display method of the analysis result according to the user's emotion. Emotion estimation may be realized using an emotion engine or generative AI, such as a text generation AI (e.g., LLM) or multimodal generative AI, but is not limited to these examples. Some or all of the above-described processing in the analysis unit may be performed using AI or without using AI. For example, the analysis unit may input the user's facial expression data to a generative AI and have the generative AI estimate emotion. Specifically, the analysis unit simultaneously acquires the user's facial image (e.g., 128×128×3 pixel RGB tensor), voice waveform data (e.g., 1-second time-series array sampled at 16 kHz), and biometric sensor data such as heart rate time-series (e.g., 60-element vector sampled at 1 Hz) and skin conductance values (e.g., 60-element vector sampled at 1 Hz), and inputs these to an emotion estimation AI model. The analysis unit applies a convolutional neural network (CNN) to the facial image to extract facial features (e.g., degree of mouth corner lift, frown lines, eye opening, etc.). For voice data, it extracts acoustic features such as Mel-frequency cepstral coefficients (MFCC), pitch, and formants, and inputs them to a recurrent neural network (RNN) or Transformer-based voice emotion recognition model. For biometric data, it extracts features such as heart rate variability and skin conductance fluctuation patterns and inputs them to a multilayer perceptron (MLP) or time-series model. The AI model integrates these multimodal features and outputs emotion classes (e.g., relaxed, tense, stressed, joyful) and emotion scores (e.g., continuous values from 0 to 1). Examples of input include “128×128×3 facial image tensor,” “1-second voice waveform array,” “60-element heart rate vector,” and “60-element skin conductance vector.” Examples of output from AI include “emotion class: relaxed, emotion score: 0.85,” “emotion class: tense, emotion score: 0.30,” etc. The analysis unit automatically adjusts the display method of the analysis result only when the output emotion score exceeds a certain threshold (e.g., 0.7 or higher for relaxed, 0.3 or lower for tense), displaying detailed analysis results when the user is relaxed and concise displays, color scheme/font size changes, or suppression of animation effects to optimize the user experience when the user is tense. In subsequent processing, the emotion estimation result is sent to the instruction unit or confirmation unit and used for interaction adjustment to optimize the user experience and reduce stress. As a technical effect, the analysis unit can estimate the user's emotional state with high accuracy from multimodal data and present the analysis result in the optimal display method, thereby reducing user stress and discomfort and improving the acceptance and behavioral change rate of the analysis result. This also improves the determination accuracy in the analysis unit and the quality of personalized instruction generation in the instruction unit. Application fields include self-care support in general households, stress-free remote monitoring for nursing facilities and the elderly, reduction of patient burden in dental clinics, and tooth brushing instruction for children in educational settings. Furthermore, the emotion estimation AI model is optimized for each user through transfer learning and continual learning, improving accuracy over long-term care cycles.

The analysis unit can improve analysis accuracy during analysis based on past analysis data. For example, the analysis unit can correct the current analysis result based on past analysis data. The analysis unit can refer to past analysis data, compare it with the current analysis result, and improve the accuracy of abnormality detection. The analysis unit can also optimize the analysis algorithm by referring to past analysis data. For example, the analysis unit can adjust the parameters of the analysis algorithm based on past analysis data to improve analysis accuracy. Furthermore, the analysis unit can improve the accuracy of abnormality detection by utilizing past analysis data. For example, the analysis unit can adjust the criteria for abnormality detection based on past analysis data to improve the accuracy of abnormality detection. Thus, by referring to past analysis data, the analysis unit can improve analysis accuracy. Some or all of the above-described processing in the analysis unit may be performed using AI or without using AI. For example, the analysis unit may input past analysis data to a generative AI and have the generative AI improve analysis accuracy. Specifically, the analysis unit manages past analysis data accumulated for each user (e.g., results of tartar detection for the past 100 times, unbrushed area probability maps, abnormality detection labels, image quality indicators, etc.) as a time-series database. The analysis unit inputs these history data as time-series vectors or tensors (e.g., history of unbrushed area probability maps of 100×224×224) to an AI model, and uses recurrent neural networks (RNN), time-series autoregressive models, or time-series Transformers to extract past trends, periodicity, and abnormality occurrence patterns. Examples of input to AI include “tensor of unbrushed area probability maps for the past 100 times,” “time-series array of abnormality detection labels,” and “history vector of image quality indicators.” Examples of output from AI include “correction value for current analysis result,” “automatic adjustment value for abnormality detection threshold,” and “optimized set of algorithm parameters.” The analysis unit uses these outputs to perform correction processing on the current analysis result reflecting past trends (e.g., dynamic change of abnormality detection threshold, automatic adjustment of detection sensitivity, suppression of false detection patterns). Furthermore, the analysis unit compares past analysis data with the current analysis result and automatically optimizes the parameters of the abnormality detection algorithm (e.g., threshold, weight coefficients, feature selection) to continuously improve analysis accuracy. In subsequent processing, the corrected analysis result is sent to the instruction unit or confirmation unit and used for feedback to the user or optimization of care instructions. As a technical effect, the analysis unit, unlike conventional static threshold settings or simple image analysis, learns and utilizes past analysis data in a time-series manner to achieve improved abnormality detection accuracy, reproducibility, and reduced false detection rate. This enables individual optimization for each user and long-term optimization of the care cycle, greatly improving the reliability of the entire system and the quality of the user experience. Application fields include self-care support in general households, long-term monitoring for nursing facilities and the elderly, time-series screening in dental clinics, and continuous tooth brushing instruction in educational settings.

The analysis unit can add a function to detect abnormalities inside the mouth during analysis and issue a warning. For example, when an abnormality inside the mouth is detected, the analysis unit can issue a warning to the user. The analysis unit can display different warning messages according to the type of abnormality. The analysis unit can also propose countermeasures when an abnormality is detected. For example, when an abnormality is detected, the analysis unit can propose specific countermeasures to the user, such as “Tartar has been found. Please consult a dentist.” Thus, by detecting abnormalities inside the mouth early and issuing warnings to the user, the analysis unit enables early response. Some or all of the above-described processing in the analysis unit may be performed using AI or without using AI. For example, the analysis unit may input abnormality detection data to a generative AI and have the generative AI generate warning messages. Specifically, the analysis unit inputs oral cavity image tensors (e.g., 224×224×3 pixels) obtained from the imaging unit to image recognition models such as convolutional neural networks (CNN) or Vision Transformer to detect abnormal regions such as tartar, inflammation, bleeding, and cavities with high accuracy. Examples of input to AI include “224×224×3 oral cavity image tensor,” “abnormality detection history vector,” and “user health history data.” Examples of output from AI include “binary mask of abnormal regions (224×224),” “abnormality type label (e.g., tartar, inflammation, bleeding),” and “warning message text (e.g., Tartar has been found. Please consult a dentist).” Based on the abnormality detection result, the analysis unit highlights abnormal areas in red or with blinking display on the user interface and automatically generates different warning messages and countermeasures for each abnormality type (e.g., dental visit recommendation for tartar, gargling instruction for inflammation, etc.). Large language models or multimodal generative models are used for warning message generation, and the content can be personalized according to the user's age, health history, and past abnormality occurrence trends. In subsequent processing, warning messages and countermeasures are sent to the instruction unit or confirmation unit and used for real-time notification and care instructions to the user. As a technical effect, the analysis unit, unlike conventional human visual confirmation or simple warning display, greatly improves the rate of early detection and early response to abnormalities through highly accurate abnormality detection and personalized warning generation by AI. This enables reduction of health risks for users, optimization of medical institution visits, and improvement of system reliability. Application fields include self-care support in general households, abnormality monitoring for nursing facilities and the elderly, pre-screening in dental clinics, and health instruction in educational settings.

The analysis unit can estimate the user's emotion and determine the priority of the analysis result based on the estimated emotion of the user. For example, the analysis unit can capture the user's facial expression with a camera and estimate the user's emotion using an emotion estimation algorithm. For example, the analysis unit can calculate an emotion score based on changes in the user's facial expression and determine whether the user is relaxed or tense. The analysis unit may also record the user's voice and estimate the user's emotion using voice analysis technology. For example, the analysis unit can analyze the tone and speed of the user's voice and calculate an emotion score. Furthermore, the analysis unit can collect the user's biometric data (heart rate and skin conductance) with sensors and estimate emotion using an emotion estimation algorithm. For example, the analysis unit can calculate an emotion score based on fluctuations in the user's heart rate. Thus, the analysis unit can determine the priority of the analysis result according to the user's emotion. Emotion estimation may be realized using an emotion engine or generative AI, such as a text generation AI (e.g., LLM) or multimodal generative AI, but is not limited to these examples. Some or all of the above-described processing in the analysis unit may be performed using AI or without using AI. For example, the analysis unit may input the user's facial expression data to a generative AI and have the generative AI estimate emotion. Specifically, the analysis unit simultaneously acquires the user's facial image (e.g., 128×128×3 pixel RGB tensor), voice waveform data (e.g., 1-second time-series array sampled at 16 kHz), and biometric sensor data such as heart rate time-series (e.g., 60-element vector sampled at 1 Hz) and skin conductance values (e.g., 60-element vector sampled at 1 Hz), and inputs these to an emotion estimation AI model. The analysis unit applies a convolutional neural network (CNN) to the facial image to extract facial features (e.g., degree of mouth corner lift, frown lines, eye opening, etc.). For voice data, it extracts acoustic features such as Mel-frequency cepstral coefficients (MFCC), pitch, and formants, and inputs them to a recurrent neural network (RNN) or Transformer-based voice emotion recognition model. For biometric data, it extracts features such as heart rate variability and skin conductance fluctuation patterns and inputs them to a multilayer perceptron (MLP) or time-series model. The AI model integrates these multimodal features and outputs emotion classes (e.g., relaxed, tense, stressed, joyful) and emotion scores (e.g., continuous values from 0 to 1). Examples of input include “128×128×3 facial image tensor,” “1-second voice waveform array,” “60-element heart rate vector,” and “60-element skin conductance vector.” Examples of output from AI include “emotion class: relaxed, emotion score: 0.85,” “emotion class: tense, emotion score: 0.30,” etc. The analysis unit automatically adjusts the priority of the analysis result only when the output emotion score exceeds a certain threshold (e.g., 0.7 or higher for relaxed, 0.3 or lower for tense), prioritizing detailed analysis results when the user is relaxed and highlighting only important analysis results when the user is tense to optimize the user experience. In subsequent processing, the prioritized analysis results are sent to the instruction unit or confirmation unit and used for feedback to the user or optimization of care instructions. As a technical effect, the analysis unit can estimate the user's emotional state with high accuracy from multimodal data and present the analysis result with optimal priority, thereby reducing user stress and discomfort and improving the acceptance and behavioral change rate of the analysis result. This also improves the determination accuracy in the analysis unit and the quality of personalized instruction generation in the instruction unit. Application fields include self-care support in general households, stress-free remote monitoring for nursing facilities and the elderly, reduction of patient burden in dental clinics, and tooth brushing instruction for children in educational settings. Furthermore, the emotion estimation AI model is optimized for each user through transfer learning and continual learning, improving accuracy over long-term care cycles.

The analysis unit can customize the analysis result during analysis based on the user's oral health history. For example, the analysis unit can customize the analysis result based on the user's oral health history. The analysis unit can refer to the user's past health data, compare it with the current analysis result, and improve the accuracy of abnormality detection. The analysis unit can also optimize the analysis algorithm by utilizing the user's health history. For example, the analysis unit can adjust the parameters of the analysis algorithm based on the user's health history to improve analysis accuracy. Furthermore, the analysis unit can adjust the criteria for abnormality detection based on the user's health history to improve the accuracy of abnormality detection. Thus, by referring to the user's oral health history, the analysis unit can customize the analysis result and improve accuracy. Some or all of the above-described processing in the analysis unit may be performed using AI or without using AI. For example, the analysis unit may input the user's health history data to a generative AI and have the generative AI customize the analysis result. Specifically, the analysis unit manages health history data accumulated for each user (e.g., results of tartar detection for the past 100 times, unbrushed area probability maps, abnormality detection labels, oral cavity image quality indicators, dental visit history, treatment history, oral dryness score, etc.) as a time-series database. The analysis unit inputs these history data as high-dimensional tensors (e.g., history of unbrushed area probability maps of 100×224×224, array of abnormality detection labels of 100 elements, treatment history vector of 100 elements, etc.) to an AI model, and uses recurrent neural networks (RNN), time-series autoregressive models, or time-series Transformers to extract trends, periodicity, abnormality occurrence patterns, and treatment effect transitions of past health conditions. Examples of input to AI include “tensor of unbrushed area probability maps for the past 100 times,” “time-series array of abnormality detection labels,” “treatment history vector,” and “history of oral dryness scores.” Examples of output from AI include “correction value for current analysis result (e.g., automatic adjustment value for abnormality detection sensitivity),” “dynamic change value for abnormality detection threshold,” and “optimized set of algorithm parameters (e.g., automatic adjustment of feature weight coefficients).” The analysis unit uses these outputs to perform correction processing on the current analysis result reflecting past health history (e.g., dynamic change of abnormality detection threshold, automatic adjustment of detection sensitivity, suppression of false detection patterns, focused monitoring of specific areas based on treatment history). Furthermore, the analysis unit compares past health history with the current analysis result and automatically optimizes the parameters of the abnormality detection algorithm (e.g., threshold, weight coefficients, feature selection) to continuously improve analysis accuracy. In subsequent processing, the corrected analysis result is sent to the instruction unit or confirmation unit and used for feedback to the user or optimization of care instructions. As a technical effect, the analysis unit, unlike conventional static threshold settings or simple image analysis, learns and utilizes user-specific health history data in a time-series manner to achieve improved abnormality detection accuracy, reproducibility, reduced false detection rate, and individual optimization. This enables long-term optimization of the care cycle for each user, focused monitoring based on treatment history, and early detection of recurrence risk, greatly improving the reliability of the entire system and the quality of the user experience. Application fields include self-care support in general households, long-term monitoring for nursing facilities and the elderly, time-series screening in dental clinics, post-treatment follow-up, and continuous tooth brushing instruction in educational settings.

The analysis unit can provide the analysis result during analysis based on the user's dietary habits and lifestyle. For example, the analysis unit can customize the analysis result by considering the user's dietary habits. The analysis unit can refer to the type and frequency of the user's meals, compare it with the current analysis result, and improve the accuracy of abnormality detection. The analysis unit can also optimize the analysis result by referring to the user's lifestyle. For example, the analysis unit can adjust the parameters of the analysis algorithm based on the user's exercise habits or sleep habits to improve analysis accuracy. Furthermore, the analysis unit can adjust the criteria for abnormality detection based on the user's dietary habits and lifestyle to improve the accuracy of abnormality detection. Thus, by considering the user's dietary habits and lifestyle, the analysis unit can provide more appropriate analysis results. Some or all of the above-described processing in the analysis unit may be performed using AI or without using AI. For example, the analysis unit may input the user's dietary habits and lifestyle data to a generative AI and have the generative AI provide the analysis result. Specifically, the analysis unit manages dietary habit data accumulated for each user (e.g., meal records for the past 90 days, intake frequency vectors for each food category, history of nutrient intake such as sugar, acidity, fiber, etc.) and lifestyle data (e.g., daily exercise time, sleep time, time-series array of bedtime and wake-up time, presence or absence of smoking/drinking habits, etc.) as high-dimensional tensors or vectors. The analysis unit inputs these dietary habit and lifestyle data to an AI model (e.g., multilayer perceptron, time-series autoregressive model, Transformer-based multimodal integration model, etc.) and statistically and machine-learn the impact of meal content and lifestyle rhythm fluctuations on the risk of tartar, unbrushed areas, and oral abnormalities. Examples of input to AI include “90×10 tensor of food category intake frequency (e.g., sugar, acidity, dairy, vegetables, meat, etc.),” “90-element vector of daily sleep time,” “90-element vector of exercise time,” and “binary label of smoking/drinking habits.” Examples of output from AI include “automatic adjustment value for abnormality detection sensitivity (e.g., lower cavity detection threshold when sugar intake is high),” “optimized set of analysis algorithm parameters (e.g., automatic adjustment of feature weight coefficients),” and “abnormality risk score (e.g., 0.75).” The analysis unit uses these outputs to perform correction processing on the current oral image analysis result reflecting the influence of dietary habits and lifestyle (e.g., dynamic change of abnormality detection threshold, automatic adjustment of detection sensitivity, suppression of false detection patterns). Furthermore, the analysis unit learns the time-series relationship between changes in dietary habits/lifestyle and the occurrence of oral abnormalities, and can perform future risk prediction and automatic selection of key monitoring areas. In subsequent processing, the corrected analysis result and risk score are sent to the instruction unit or confirmation unit and used for generating personalized care instructions and lifestyle improvement advice for the user. As a technical effect, the analysis unit, unlike conventional static image analysis or uniform threshold settings, learns and utilizes user-specific dietary habit and lifestyle data multidimensionally to achieve improved abnormality detection accuracy, reproducibility, reduced false detection rate, and individual optimization. This enables long-term optimization of the care cycle for each user and early detection/prevention of recurrence risk through lifestyle improvement, greatly improving the reliability of the entire system and the quality of the user experience. Application fields include self-care support in general households, health management for lifestyle disease prevention, long-term monitoring for nursing facilities and the elderly, lifestyle guidance collaboration in dental clinics, and dietary education/tooth brushing instruction in educational settings.

The instruction unit can estimate the user's emotion and adjust the expression method of the instructions based on the estimated emotion of the user. For example, the instruction unit can capture the user's facial expression with a camera and estimate the user's emotion using an emotion estimation algorithm. For example, the instruction unit can calculate an emotion score based on changes in the user's facial expression and determine whether the user is relaxed or tense. The instruction unit may also record the user's voice and estimate the user's emotion using voice analysis technology. For example, the instruction unit can analyze the tone and speed of the user's voice and calculate an emotion score. Furthermore, the instruction unit can collect the user's biometric data (heart rate and skin conductance) with sensors and estimate emotion using an emotion estimation algorithm. For example, the instruction unit can calculate an emotion score based on fluctuations in the user's heart rate. Thus, the instruction unit can adjust the expression method of the instructions according to the user's emotion. Emotion estimation may be realized using an emotion engine or generative AI, such as a text generation AI (e.g., LLM) or multimodal generative AI, but is not limited to these examples. Some or all of the above-described processing in the instruction unit may be performed using AI or without using AI. For example, the instruction unit may input the user's facial expression data to a generative AI and have the generative AI estimate emotion. Specifically, the instruction unit simultaneously acquires the user's facial image (e.g., 128×128×3 pixel RGB tensor), voice waveform data (e.g., 1-second time-series array sampled at 16 kHz), and biometric sensor data such as heart rate time-series (e.g., 60-element vector sampled at 1 Hz) and skin conductance values (e.g., 60-element vector sampled at 1 Hz), and inputs these to an emotion estimation AI model. The instruction unit applies a convolutional neural network (CNN) to the facial image to extract facial features (e.g., degree of mouth corner lift, frown lines, eye opening, etc.). For voice data, it extracts acoustic features such as Mel-frequency cepstral coefficients (MFCC), pitch, and formants, and inputs them to a recurrent neural network (RNN) or Transformer-based voice emotion recognition model. For biometric data, it extracts features such as heart rate variability and skin conductance fluctuation patterns and inputs them to a multilayer perceptron (MLP) or time-series model. The AI model integrates these multimodal features and outputs emotion classes (e.g., relaxed, tense, stressed, joyful) and emotion scores (e.g., continuous values from 0 to 1). Examples of input include “128×128×3 facial image tensor,” “1-second voice waveform array,” “60-element heart rate vector,” and “60-element skin conductance vector.” Examples of output from AI include “emotion class: relaxed, emotion score: 0.85,” “emotion class: tense, emotion score: 0.30,” etc. The instruction unit automatically adjusts the expression method of the instructions only when the output emotion score exceeds a certain threshold (e.g., 0.7 or higher for relaxed, 0.3 or lower for tense), providing detailed instruction sentences, polite expressions, and animated guide displays when the user is relaxed, and concise instruction sentences, calm color schemes/font size changes, and suppression of animation effects to optimize the user experience when the user is tense. In subsequent processing, the emotion estimation result is sent to the analysis unit or confirmation unit and used for interaction adjustment to optimize the user experience and reduce stress. As a technical effect, the instruction unit can estimate the user's emotional state with high accuracy from multimodal data and present instructions in the optimal expression method, thereby reducing user stress and discomfort and improving the acceptance and behavioral change rate of the instruction content. This greatly improves the quality of personalized instruction generation and care continuity by the instruction unit. Application fields include self-care support in general households, stress-free remote guidance for nursing facilities and the elderly, patient education in dental clinics, and tooth brushing instruction for children in educational settings. Furthermore, the emotion estimation AI model is optimized for each user through transfer learning and continual learning, improving accuracy over long-term care cycles.

The instruction unit can present specific improvement points during instruction based on the user's brushing habits. For example, the instruction unit can analyze the user's brushing habits and present specific improvement points. The instruction unit can analyze the user's brushing data, identify areas with frequent unbrushed spots or brushing patterns, and present improvement points based on that analysis. The instruction unit can also propose optimal brushing methods according to the user's brushing habits. For example, the instruction unit can propose specific brushing methods to prevent unbrushed areas based on the user's brushing data. Furthermore, the instruction unit can present methods to prevent unbrushed areas according to the user's brushing habits. For example, the instruction unit can present specific methods to prevent unbrushed areas based on the user's brushing data. Thus, by considering the user's brushing habits, the instruction unit can present specific improvement points and prevent unbrushed areas. Some or all of the above-described processing in the instruction unit may be performed using AI or without using AI. For example, the instruction unit may input the user's brushing data to a generative AI and have the generative AI present improvement points. Specifically, the instruction unit manages brushing history data (e.g., array of unbrushed area labels for the past 30 times, time-series vector of brushing pressure sensor values, multidimensional vectors of brushing time, brush angle, movement patterns, etc.) as a time-series database. The instruction unit inputs these brushing data to AI models such as recurrent neural networks (RNN), autoregressive models, or time-series Transformers to automatically extract areas with frequent unbrushed spots or brushing habits (e.g., unbrushed areas on the lower right molar, weak brush pressure, short brushing time, etc.). Examples of input to AI include “array of unbrushed area labels for 30 times,” “time-series vector of brushing pressure for 30 times,” and “vector of brushing time for 30 times.” Examples of output from AI include “list of improvement points (e.g., lower left molar: adjust brush angle, upper right incisor: extend brushing time),” “cluster label of brushing patterns,” and “automatic selection of key instruction areas.” Based on these outputs, the instruction unit generates personalized improvement points for each user (e.g., “There are many unbrushed areas on the lower left molar, so try changing the angle of the brush,” “Extend brushing time for the upper right incisor by 5 seconds,” etc.) and presents them as user interface, voice guide, or brushing guide video. Furthermore, the instruction unit can learn the user's brushing habits and improvement history to personalize instruction content and expression methods for future instructions. As a technical effect, the instruction unit, unlike conventional static instruction displays or simple warning sounds, can greatly improve the user's rate of improvement in unbrushed areas and care continuity through high-dimensional feature extraction and personalized instruction generation by AI. Furthermore, by integrally analyzing multidimensional data such as the user's brushing history and emotional state, the instruction content and expression methods can be dynamically optimized to enhance the quality of the user experience. Application fields include self-care support in general households, remote guidance for nursing facilities and the elderly, patient education in dental clinics, and tooth brushing instruction in educational settings.

The instruction unit can provide different instruction content according to the condition of the user's oral cavity at the time of instruction. For example, the instruction unit can provide normal instruction content when the user's oral cavity is healthy. For instance, the instruction unit can provide standard instruction content when there are no abnormalities in the user's oral cavity. Additionally, the instruction unit can provide detailed instruction content when there are abnormalities in the user's oral cavity. For example, the instruction unit can instruct detailed care methods when abnormalities are present in the user's oral cavity. Furthermore, the instruction unit can provide instruction content that considers humidity when the user's oral cavity is dry. For example, the instruction unit can instruct specific methods to maintain humidity when the user's oral cavity is dry. Thus, by providing instruction content according to the condition of the user's oral cavity, the instruction unit enables appropriate care. Some or all of the above-described processing in the instruction unit may be performed using AI or without using AI. For example, the instruction unit can input the user's oral condition data into a generative AI and have the generative AI generate the instruction content. Specifically, the instruction unit receives oral health condition data (e.g., tartar detection status, unbrushed area probability map, dryness score, etc.) and humidity/temperature sensor values (e.g., humidity 65% RH, temperature 36.5° C.) from the analysis unit as input. If the health condition is determined to be “no abnormality,” the instruction unit automatically generates standard care instructions (e.g., normal brushing method, standard brushing time, general precautions). If the health condition is determined to be “abnormal” (e.g., tartar detected, signs of inflammation, high dryness), the instruction unit automatically generates detailed care instructions (e.g., high-precision brushing guide, focused care for specific areas, rinsing/moisturizing instructions, avoidance of brushing inflamed areas, etc.). If humidity is low, the instruction unit presents additional instructions for oral moisturizing methods (e.g., recommendation of rinsing, hydration, use of moisturizing gel, etc.). Examples of input to AI include “health condition label: no abnormality,” “humidity value: 65% RH,” “dryness score: 0.8,” etc. Examples of output from AI include “instruction content: standard care,” “instruction content: high-precision care/moisturizing recommendation,” “focused care area label,” etc. The instruction unit can also learn each user's health condition history and past care instruction history to personalize future instruction content and expression methods. As a technical effect, unlike conventional uniform instruction content, the instruction unit achieves highly accurate care instructions for abnormal areas and dry conditions by dynamically optimizing instruction content based on AI health condition determination and environmental sensing. This improves care quality and care continuity for each user and enhances the overall reliability of the system. Application fields include self-care support in general households, remote monitoring in nursing facilities, pre-screening in dental clinics, and tooth brushing guidance in educational settings.

The instruction unit can estimate the user's emotion and adjust the level of detail of instructions based on the estimated emotion. For example, the instruction unit can capture the user's facial expression with a camera and estimate the user's emotion using an emotion estimation algorithm. For instance, the instruction unit can calculate an emotion score based on changes in the user's facial expression and determine whether the user is relaxed or tense. Additionally, the instruction unit can record the user's voice and estimate the user's emotion using voice analysis technology. For example, the instruction unit can analyze the tone and speed of the user's voice to calculate an emotion score. Furthermore, the instruction unit can collect the user's biometric data (heart rate and skin conductance) with sensors and estimate emotion using an emotion estimation algorithm. For example, the instruction unit can calculate an emotion score based on fluctuations in the user's heart rate. Thus, the instruction unit can adjust the level of detail of instructions according to the user's emotion. Emotion estimation may be implemented using, for example, an emotion engine or generative AI with emotion estimation functions. Generative AI may include text generation AI (e.g., LLM) or multimodal generative AI, but is not limited to these examples. Some or all of the above-described processing in the instruction unit may be performed using AI or without using AI. For example, the instruction unit can input the user's facial expression data into a generative AI and have the generative AI estimate the emotion. Specifically, the instruction unit simultaneously acquires the user's facial image (e.g., 128×128×3 pixel RGB tensor), voice waveform data (e.g., 1-second time series array sampled at 16 kHz), and biometric sensor data such as heart rate time series (e.g., 60-element vector sampled at 1 Hz) and skin conductance values (e.g., 60-element vector sampled at 1 Hz), and inputs these into an emotion estimation AI model. The instruction unit applies a convolutional neural network (CNN) to the facial image to extract facial features (e.g., degree of mouth corner lift, frown lines, degree of eye opening, etc.). For voice data, it extracts acoustic features such as Mel-frequency cepstral coefficients (MFCC), pitch, and formants, and inputs them into a recurrent neural network (RNN) or Transformer-based speech emotion recognition model. For biometric data, it extracts features such as heart rate variability and skin conductance fluctuation patterns and inputs them into a multilayer perceptron (MLP) or time series model. The AI model integrates these multimodal features and outputs emotion classes (e.g., relaxed, tense, stressed, joyful, etc.) and emotion scores (e.g., continuous values from 0 to 1). Examples of input to AI include “128×128×3 facial image tensor,” “1-second voice waveform array,” “60-element heart rate vector,” “60-element skin conductance vector,” etc. Examples of output from AI include “emotion class: relaxed, emotion score: 0.85,” “emotion class: tense, emotion score: 0.30,” etc. The instruction unit automatically adjusts the level of detail of instructions only when the output emotion score exceeds a certain threshold (e.g., 0.7 or higher for relaxed, 0.3 or lower for tense), presenting detailed instruction text, polite explanations, diagrams, or video guides when the user is relaxed, and concise instruction text, key points only, and suppressed animation effects when the user is tense, thereby optimizing the user experience through detail control. In subsequent processing, the emotion estimation result is sent to the analysis unit or confirmation unit and used for interaction adjustment to optimize user experience and reduce stress. As a technical effect, the instruction unit accurately estimates the user's emotional state from multimodal data and presents instructions at the optimal level of detail, thereby reducing user stress and discomfort and improving the acceptability of instruction content and behavior change rate. This greatly improves the quality of personalized instruction generation and care continuity by the instruction unit. Application fields include self-care support in general households, stress-free remote guidance for nursing facilities and the elderly, patient education in dental clinics, and tooth brushing guidance for children in educational settings. Furthermore, the emotion estimation AI model is optimized for each user through transfer learning and continual learning, improving accuracy over long-term care cycles.

The instruction unit can provide different instruction content according to the user's age and gender at the time of instruction. For example, the instruction unit can provide appropriate instruction content according to the user's age. For instance, the instruction unit can customize tooth care methods according to the user's age. Additionally, the instruction unit can provide optimal instruction content according to the user's gender. For example, the instruction unit can customize tooth care methods according to the user's gender. Furthermore, the instruction unit can provide customized instruction content by considering both the user's age and gender. For example, the instruction unit can optimize tooth care methods according to the user's age and gender. Thus, by providing instruction content according to the user's age and gender, the instruction unit enables appropriate care. Some or all of the above-described processing in the instruction unit may be performed using AI or without using AI. For example, the instruction unit can input the user's age and gender data into a generative AI and have the generative AI generate the instruction content.

The instruction unit can propose brushing methods according to the type of toothbrush used by the user at the time of instruction. For example, the instruction unit can analyze the type of toothbrush used by the user and propose the optimal brushing method. For instance, the instruction unit can provide details of brushing methods according to the type of toothbrush used by the user. Additionally, the instruction unit can propose methods to prevent unbrushed areas according to the type of toothbrush used by the user. For example, the instruction unit can propose specific methods to prevent unbrushed areas by considering the type of toothbrush used by the user. Thus, by proposing the optimal brushing method according to the type of toothbrush used by the user, the instruction unit can prevent unbrushed areas. Some or all of the above-described processing in the instruction unit may be performed using AI or without using AI. For example, the instruction unit can input the user's toothbrush type data into a generative AI and have the generative AI propose brushing methods.

The confirmation unit can estimate the user's emotion and adjust the display method of the confirmation result based on the estimated emotion. For example, the confirmation unit can capture the user's facial expression with a camera and estimate the user's emotion using an emotion estimation algorithm. For instance, the confirmation unit can calculate an emotion score based on changes in the user's facial expression and determine whether the user is relaxed or tense. Additionally, the confirmation unit can record the user's voice and estimate the user's emotion using voice analysis technology. For example, the confirmation unit can analyze the tone and speed of the user's voice to calculate an emotion score. Furthermore, the confirmation unit can collect the user's biometric data (heart rate and skin conductance) with sensors and estimate emotion using an emotion estimation algorithm. For example, the confirmation unit can calculate an emotion score based on fluctuations in the user's heart rate. Thus, the confirmation unit can adjust the display method of the confirmation result according to the user's emotion. Emotion estimation may be implemented using, for example, an emotion engine or generative AI with emotion estimation functions. Generative AI may include text generation AI (e.g., LLM) or multimodal generative AI, but is not limited to these examples. Some or all of the above-described processing in the confirmation unit may be performed using AI or without using AI. For example, the confirmation unit can input the user's facial expression data into a generative AI and have the generative AI estimate the emotion.

The confirmation unit can add a function to display areas of unbrushed areas at the time of confirmation. For example, the confirmation unit can highlight areas of unbrushed areas in red. For instance, by highlighting areas of unbrushed areas in red, the confirmation unit enables the user to visually confirm them easily. Additionally, the confirmation unit can enlarge the display of areas of unbrushed areas. For example, by enlarging the display of areas of unbrushed areas, the confirmation unit enables the user to confirm them in detail. Furthermore, the confirmation unit can make areas of unbrushed areas blink. For example, by making areas of unbrushed areas blink, the confirmation unit enables the user to visually confirm them easily. Thus, by highlighting areas of unbrushed areas, the confirmation unit makes it easier for the user to confirm unbrushed areas. Some or all of the above-described processing in the confirmation unit may be performed using AI or without using AI. For example, the confirmation unit can input unbrushed area data into a generative AI and have the generative AI generate the display method.

The confirmation unit can add a function to provide improvement points for the user's brushing method at the time of confirmation. For example, the confirmation unit can provide specific feedback on improvement points for the user's brushing method. For instance, the confirmation unit can analyze the user's brushing data, identify areas with many unbrushed areas or brushing patterns, and present improvement points based on that analysis. Additionally, the confirmation unit can analyze the user's brushing habits and present improvement points. For example, the confirmation unit can present specific methods to prevent unbrushed areas based on the user's brushing data. Furthermore, the confirmation unit can present improvement points for the user's brushing method in a video. For example, the confirmation unit can present specific methods to prevent unbrushed areas in a video based on the user's brushing data. Thus, by providing feedback on improvement points for the user's brushing method, the confirmation unit enables the user to learn appropriate brushing methods. Some or all of the above-described processing in the confirmation unit may be performed using AI or without using AI. For example, the confirmation unit can input the user's brushing data into a generative AI and have the generative AI present improvement points.

The confirmation unit can estimate the user's emotion and adjust the frequency of confirmation based on the estimated emotion. For example, the confirmation unit can capture the user's facial expression with a camera and estimate the user's emotion using an emotion estimation algorithm. For instance, the confirmation unit can calculate an emotion score based on changes in the user's facial expression and determine whether the user is relaxed or tense. Additionally, the confirmation unit can record the user's voice and estimate the user's emotion using voice analysis technology. For example, the confirmation unit can analyze the tone and speed of the user's voice to calculate an emotion score. Furthermore, the confirmation unit can collect the user's biometric data (heart rate and skin conductance) with sensors and estimate emotion using an emotion estimation algorithm. For example, the confirmation unit can calculate an emotion score based on fluctuations in the user's heart rate. Thus, the confirmation unit can adjust the frequency of confirmation according to the user's emotion. Emotion estimation may be implemented using, for example, an emotion engine or generative AI with emotion estimation functions. Generative AI may include text generation AI (e.g., LLM) or multimodal generative AI, but is not limited to these examples. Some or all of the above-described processing in the confirmation unit may be performed using AI or without using AI. For example, the confirmation unit can input the user's facial expression data into a generative AI and have the generative AI estimate the emotion.

The confirmation unit can provide confirmation methods according to the health condition of the user's oral cavity at the time of confirmation. For example, the confirmation unit can provide normal confirmation methods when the user's oral cavity is healthy. For instance, the confirmation unit can provide standard confirmation methods when there are no abnormalities in the user's oral cavity. Additionally, the confirmation unit can provide detailed confirmation methods when there are abnormalities in the user's oral cavity. For example, the confirmation unit can provide detailed confirmation methods when abnormalities are present in the user's oral cavity. Furthermore, the confirmation unit can provide confirmation methods that consider humidity when the user's oral cavity is dry. For example, the confirmation unit can provide specific methods to maintain humidity when the user's oral cavity is dry. Thus, by providing confirmation methods according to the health condition of the user's oral cavity, the confirmation unit enables appropriate care. Some or all of the above-described processing in the confirmation unit may be performed using AI or without using AI. For example, the confirmation unit can input the user's oral health condition data into a generative AI and have the generative AI provide confirmation methods.

The confirmation unit can customize the confirmation result by referring to the user's brushing history at the time of confirmation. For example, the confirmation unit can customize the confirmation result based on the user's brushing history. For instance, the confirmation unit can refer to the user's past brushing data, compare it with the current confirmation result, and improve the accuracy of abnormality detection. Additionally, the confirmation unit can optimize the confirmation algorithm by utilizing the user's brushing history. For example, the confirmation unit can adjust the parameters of the confirmation algorithm based on the user's brushing history to improve confirmation accuracy. Furthermore, the confirmation unit can adjust the abnormality detection criteria by referring to the user's brushing history. For example, the confirmation unit can adjust the abnormality detection criteria based on the user's brushing history to improve the accuracy of abnormality detection. Thus, by referring to the user's brushing history, the confirmation unit can customize the confirmation result and improve accuracy. Some or all of the above-described processing in the confirmation unit may be performed using AI or without using AI. For example, the confirmation unit can input the user's brushing history data into a generative AI and have the generative AI customize the confirmation result.

The system according to the embodiment is not limited to the above-described examples, and various modifications are possible, for example, as follows.

The analysis unit can analyze the user's dietary content and identify factors that affect dental health. For example, the analysis unit can analyze the types and frequency of foods consumed by the user and evaluate the impact of foods high in sugar or acidity on the teeth. Additionally, the analysis unit can propose dietary improvements to maintain dental health based on the user's dietary content. Furthermore, the analysis unit can monitor the relationship between the user's dietary content and dental health over the long term and evaluate the effect of dietary improvements on dental health. Thus, by considering the user's dietary content, the analysis unit can provide specific advice for maintaining dental health.

The instruction unit can customize tooth care methods by considering the user's lifestyle habits. For example, the instruction unit can analyze the user's smoking and drinking habits and propose tooth care methods based on them. Additionally, the instruction unit can optimize tooth care methods by considering the user's exercise and sleep habits. Furthermore, the instruction unit can evaluate the user's stress level and propose care methods that consider the impact of stress on dental health. Thus, by comprehensively considering the user's lifestyle habits, the instruction unit can provide more effective tooth care methods.

The confirmation unit can monitor the user's dental health over the long term and provide regular reports. For example, the confirmation unit can regularly evaluate the user's dental health and summarize trends such as tartar accumulation and unbrushed areas in a report. Additionally, the confirmation unit can display changes in the user's dental health over time and indicate improvement points and cautions. Furthermore, the confirmation unit can propose a regular care schedule based on the user's dental health. Thus, the confirmation unit can provide support for the user to perform effective dental care at home.

The analysis unit can analyze the bacterial balance in the oral cavity to evaluate the user's dental health. For example, the analysis unit can analyze samples collected from the user's oral cavity and identify the types and quantities of bacteria. Additionally, the analysis unit can monitor changes in bacterial balance and identify factors that affect dental health. Furthermore, the analysis unit can propose methods to improve bacterial balance and provide advice for appropriate oral care. Thus, by considering the bacterial balance in the user's oral cavity, the analysis unit can provide specific advice for maintaining dental health.

The instruction unit can propose how to select dental care products based on the user's dental health. For example, the instruction unit can evaluate the user's dental condition and propose the optimal toothpaste or toothbrush based on that evaluation. Additionally, the instruction unit can propose how to use auxiliary care products such as floss or mouthwash according to the user's dental health. Furthermore, the instruction unit can recommend the use of care products containing specific ingredients based on the user's dental health. Thus, the instruction unit can support the user in selecting products for effective dental care at home.

The analysis unit can estimate the user's emotion and adjust the display method of the analysis result based on the estimated emotion. For example, the analysis unit can capture the user's facial expression with a camera and estimate the user's emotion using an emotion estimation algorithm. If the user is relaxed, the analysis result can be displayed in detail, and if the user is tense, it can be displayed concisely. Additionally, the analysis unit can record the user's voice and estimate emotion using voice analysis technology. Furthermore, the analysis unit can collect the user's biometric data (heart rate and skin conductance) with sensors and estimate emotion using an emotion estimation algorithm. Thus, the analysis unit can adjust the display method of the analysis result according to the user's emotion.

The instruction unit can estimate the user's emotion and adjust the expression method of instructions based on the estimated emotion. For example, the instruction unit can capture the user's facial expression with a camera and estimate the user's emotion using an emotion estimation algorithm. If the user is relaxed, detailed instructions can be provided, and if the user is tense, concise instructions can be provided. Additionally, the instruction unit can record the user's voice and estimate emotion using voice analysis technology. Furthermore, the instruction unit can collect the user's biometric data (heart rate and skin conductance) with sensors and estimate emotion using an emotion estimation algorithm. Thus, the instruction unit can adjust the expression method of instructions according to the user's emotion.

The confirmation unit can estimate the user's emotion and adjust the display method of the confirmation result based on the estimated emotion. For example, the confirmation unit can capture the user's facial expression with a camera and estimate the user's emotion using an emotion estimation algorithm. If the user is relaxed, detailed confirmation results can be displayed, and if the user is tense, concise confirmation results can be displayed. Additionally, the confirmation unit can record the user's voice and estimate emotion using voice analysis technology. Furthermore, the confirmation unit can collect the user's biometric data (heart rate and skin conductance) with sensors and estimate emotion using an emotion estimation algorithm. Thus, the confirmation unit can adjust the display method of the confirmation result according to the user's emotion.

The analysis unit can estimate the user's emotion and determine the priority of the analysis result based on the estimated emotion. For example, the analysis unit can capture the user's facial expression with a camera and estimate the user's emotion using an emotion estimation algorithm. If the user is relaxed, detailed analysis results can be displayed preferentially, and if the user is tense, only important analysis results can be displayed. Additionally, the analysis unit can record the user's voice and estimate emotion using voice analysis technology. Furthermore, the analysis unit can collect the user's biometric data (heart rate and skin conductance) with sensors and estimate emotion using an emotion estimation algorithm. Thus, the analysis unit can determine the priority of the analysis result according to the user's emotion.

The instruction unit can estimate the user's emotion and adjust the level of detail of the instructions based on the estimated emotion of the user. For example, the instruction unit can capture the user's facial expression with a camera and estimate the user's emotion using an emotion estimation algorithm. If the user is relaxed, detailed instructions can be provided, and if the user is tense, concise instructions can be provided. In addition, the instruction unit can record the user's voice and estimate the emotion using voice analysis technology. Furthermore, the instruction unit can collect the user's biometric data (such as heart rate and skin electrical activity) with sensors and estimate the emotion using an emotion estimation algorithm. In this way, the instruction unit can adjust the level of detail of the instructions according to the user's emotion.

The following is a brief description of the processing flow of Example of the Embodiment.

Step 1: The imaging unit captures images of the inside of the user's mouth with a camera. The user can use a smartphone camera or a dedicated digital camera. The imaging unit can use a wide-angle lens to capture the entire inside of the mouth, and can also use a macro lens to capture specific areas in detail.

Step 2: The analysis unit analyzes the images captured by the imaging unit using AI and determines the presence of tartar or unbrushed areas. The analysis unit uses deep learning technology or neural networks to detect tartar or unbrushed areas in the images with high accuracy and identifies their locations.

Step 3: The instruction unit informs the user where to brush based on the result determined by the analysis unit. The instruction unit can visually indicate the areas of unbrushed areas by highlighting them, and can also provide brushing instructions using a voice guide.

Step 4: The confirmation unit captures the inside of the mouth again with a camera after the user has brushed their teeth according to the instructions, and AI performs confirmation again. The confirmation unit notifies the user of the timing for re-imaging and analyzes the re-captured images to confirm whether there are any unbrushed areas remaining.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned imaging unit, analysis unit, instruction unit, and confirmation unit is implemented by at least one of, for example, a smart device 14 and a data processing apparatus 12. For example, the imaging unit captures images of the inside of the mouth using a camera 42 of the smart device 14. The analysis unit is implemented, for example, by a specific processing unit 290 of the data processing apparatus 12, and analyzes the captured images to determine the presence of tartar or unbrushed areas. The instruction unit is implemented, for example, by a control unit 46A of the smart device 14, and informs the user of the areas to be brushed based on the analysis result. The confirmation unit is implemented, for example, by the specific processing unit 290 of the data processing apparatus 12, and analyzes the images captured again to confirm whether there are any unbrushed areas. The correspondence between each unit and the apparatus or control unit is not limited to the examples described above and various modifications are possible.

### Second Embodiment

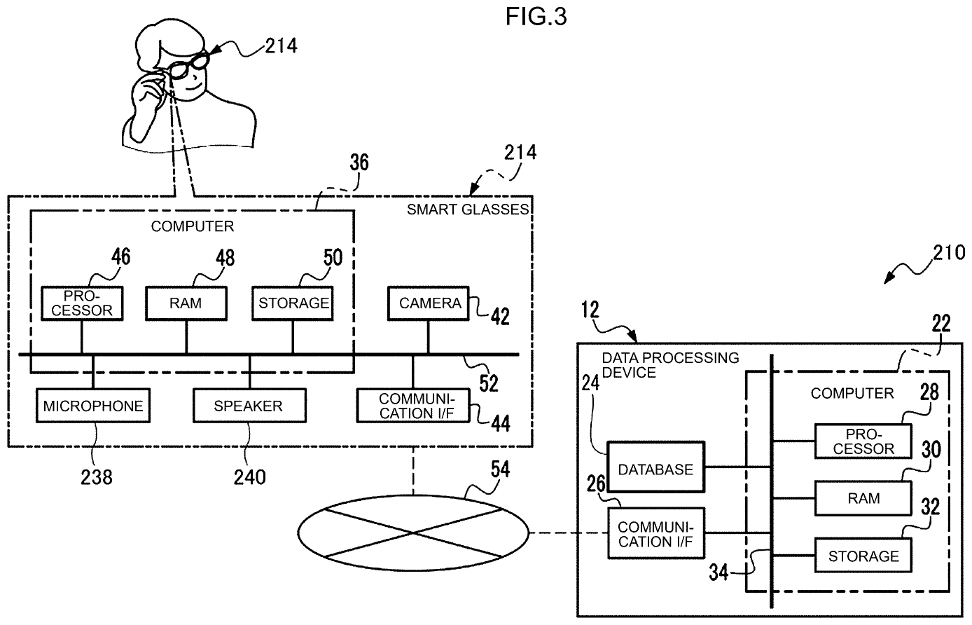

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

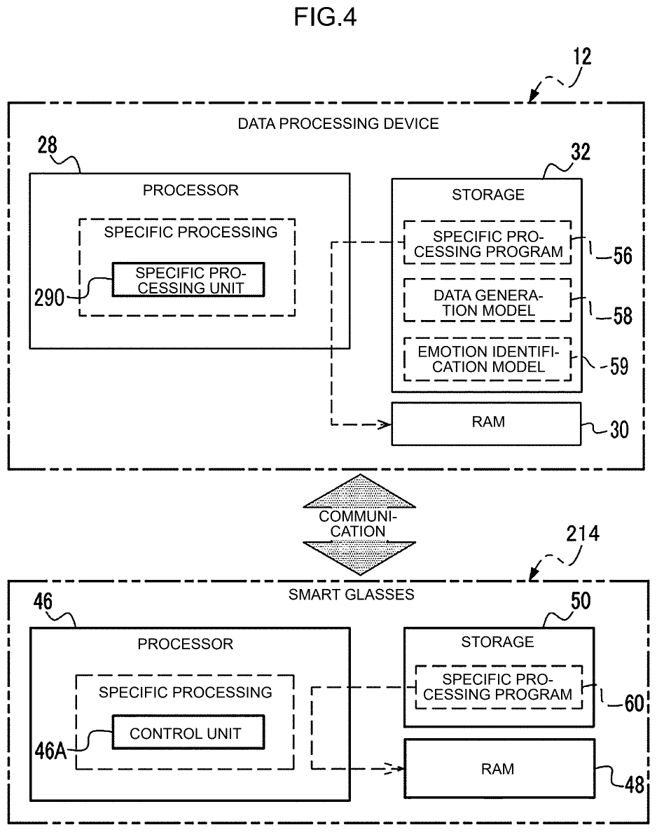

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned imaging unit, analysis unit, instruction unit, and confirmation unit is implemented by at least one of, for example, smart glasses 214 and a data processing apparatus 12. For example, the imaging unit captures images of the inside of the mouth using a camera 42 of the smart glasses 214. The analysis unit is implemented, for example, by a specific processing unit 290 of the data processing apparatus 12, and analyzes the captured images to determine the presence of tartar or unbrushed areas. The instruction unit is implemented, for example, by a control unit 46A of the smart glasses 214, and informs the user of the areas to be brushed based on the analysis result. The confirmation unit is implemented, for example, by the specific processing unit 290 of the data processing apparatus 12, and analyzes the images captured again to confirm whether there are any unbrushed areas. The correspondence between each unit and the apparatus or control unit is not limited to the examples described above and various modifications are possible.

### Third Embodiment

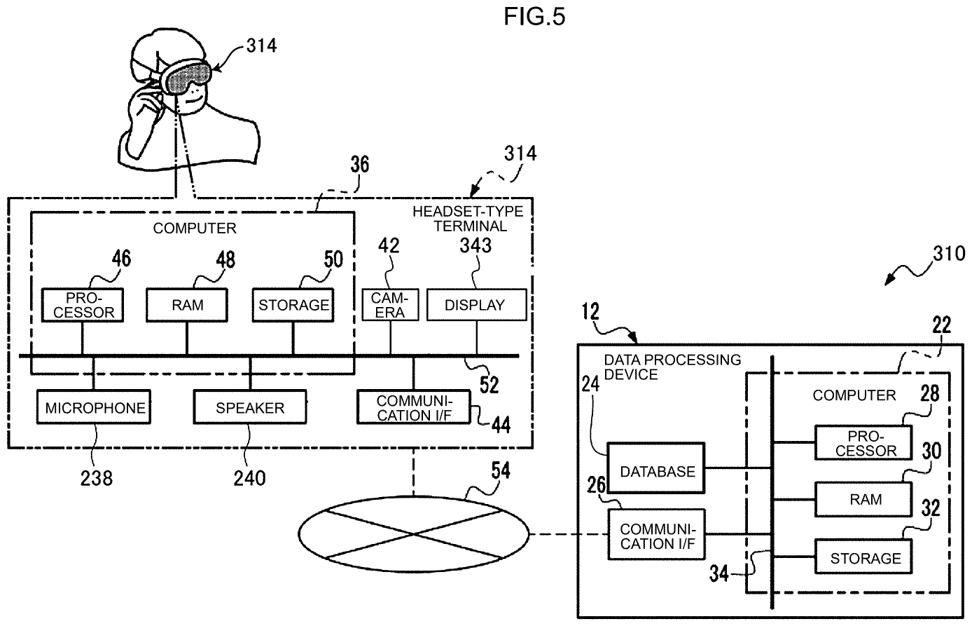

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

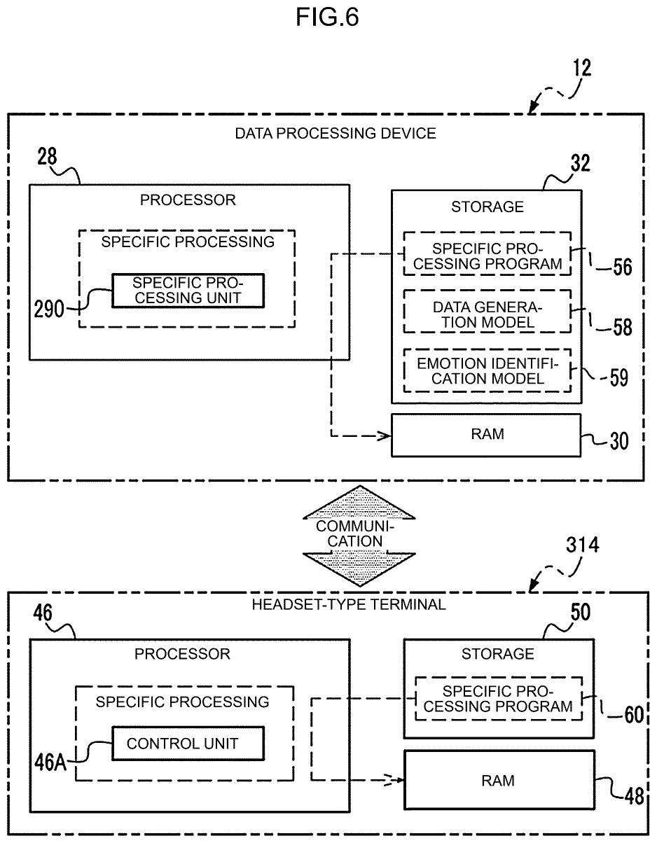

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned imaging unit, analysis unit, instruction unit, and confirmation unit is implemented by at least one of, for example, a headset-type terminal 314 and a data processing apparatus 12. For example, the imaging unit captures images of the inside of the mouth using a camera 42 of the headset-type terminal 314. The analysis unit is implemented, for example, by a specific processing unit 290 of the data processing apparatus 12, and analyzes the captured images to determine the presence of tartar or unbrushed areas. The instruction unit is implemented, for example, by a control unit 46A of the headset-type terminal 314, and informs the user of the areas to be brushed based on the analysis result. The confirmation unit is implemented, for example, by the specific processing unit 290 of the data processing apparatus 12, and analyzes the images captured again to confirm whether there are any unbrushed areas. The correspondence between each unit and the apparatus or control unit is not limited to the examples described above and various modifications are possible.

### Fourth Embodiment

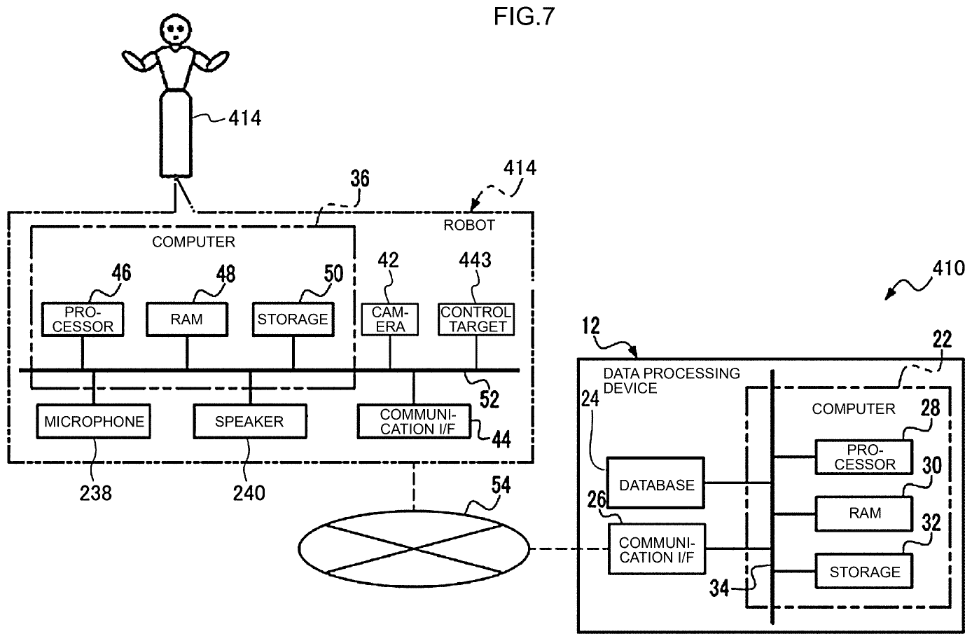

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

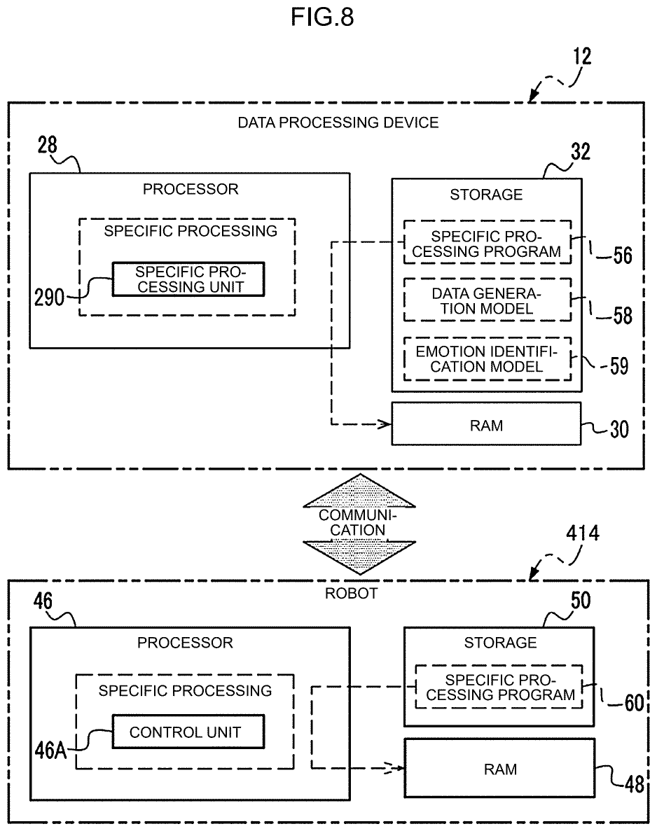

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned imaging unit, analysis unit, instruction unit, and confirmation unit is implemented by at least one of, for example, a robot 414 and a data processing apparatus 12. For example, the imaging unit captures images of the inside of the mouth using a camera 42 of the robot 414. The analysis unit is implemented, for example, by a specific processing unit 290 of the data processing apparatus 12, and analyzes the captured images to determine the presence of tartar or unbrushed areas. The instruction unit is implemented, for example, by a control unit 46A of the robot 414, and informs the user of the areas to be brushed based on the analysis result. The confirmation unit is implemented, for example, by the specific processing unit 290 of the data processing apparatus 12, and analyzes the images captured again to confirm whether there are any unbrushed areas. The correspondence between each unit and the apparatus or control unit is not limited to the examples described above and various modifications are possible.

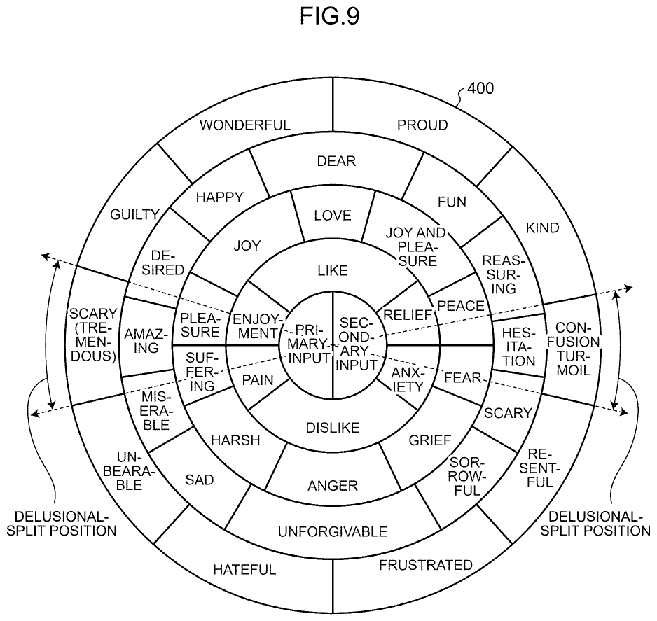

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

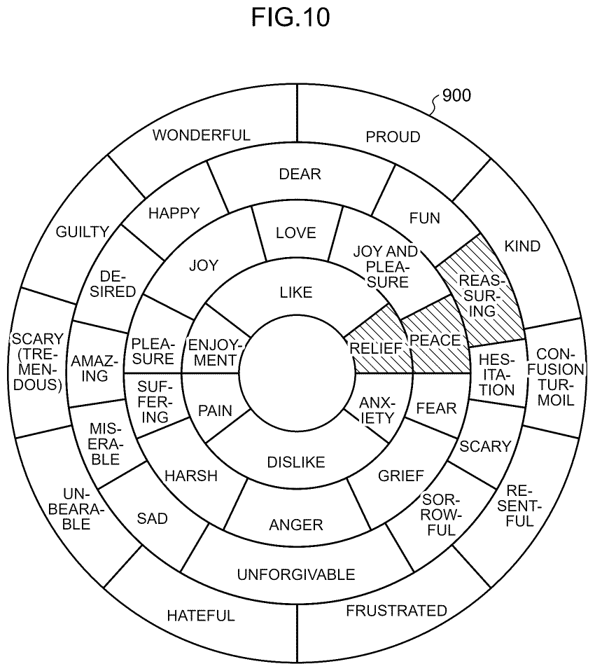

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: an imaging unit configured to capture images of the inside of a mouth; an analysis unit configured to analyze images captured by the imaging unit and determine the presence of tartar or unbrushed areas; an instruction unit configured to inform a user where to brush based on the result determined by the analysis unit; and a confirmation unit configured to capture the inside of the mouth again and perform confirmation after the user has brushed their teeth according to the instructions.
(Supplementary Note 2)The system according to Supplementary Note 1, wherein the analysis unit analyzes images using AI and determines the presence of tartar or unbrushed areas.
(Supplementary Note 3)The system according to Supplementary Note 1, wherein the instruction unit provides instructions to the user based on the result of the analysis unit.
(Supplementary Note 4)The system according to Supplementary Note 1, wherein the confirmation unit captures images of the inside of the mouth with a camera after the user has brushed their teeth according to the instructions, and confirmation is performed by AI.
(Supplementary Note 5)The system according to Supplementary Note 1, wherein the imaging unit estimates the user's emotion and adjusts the imaging timing based on the estimated emotion of the user.
(Supplementary Note 6)The system according to Supplementary Note 1, wherein the imaging unit displays a guide for focusing on specific areas inside the mouth during imaging.
(Supplementary Note 7)The system according to Supplementary Note 1, wherein the imaging unit measures humidity and temperature inside the mouth during imaging and automatically adjusts imaging conditions.
(Supplementary Note 8)The system according to Supplementary Note 1, wherein the imaging unit estimates the user's emotion and adjusts the imaging frequency based on the estimated emotion of the user.
(Supplementary Note 9)The system according to Supplementary Note 1, wherein the imaging unit selects an imaging mode according to the health condition of the user's oral cavity during imaging.
(Supplementary Note 10)The system according to Supplementary Note 1, wherein the imaging unit detects movement inside the user's oral cavity during imaging and adds a function to correct the movement.
(Supplementary Note 11)The system according to Supplementary Note 1, wherein the analysis unit estimates the user's emotion and adjusts the display method of the analysis result based on the estimated emotion of the user.
(Supplementary Note 12)The system according to Supplementary Note 1, wherein the analysis unit improves analysis accuracy during analysis based on past analysis data.
(Supplementary Note 13)The system according to Supplementary Note 1, wherein the analysis unit detects abnormalities inside the mouth during analysis and adds a function to issue a warning.
(Supplementary Note 14)The system according to Supplementary Note 1, wherein the analysis unit estimates the user's emotion and determines the priority of the analysis result based on the estimated emotion of the user.
(Supplementary Note 15)The system according to Supplementary Note 1, wherein the analysis unit customizes the analysis result during analysis based on the user's oral health history.
(Supplementary Note 16)The system according to Supplementary Note 1, wherein the analysis unit provides the analysis result during analysis based on the user's dietary habits and lifestyle.
(Supplementary Note 17)The system according to Supplementary Note 1, wherein the instruction unit estimates the user's emotion and adjusts the expression method of the instructions based on the estimated emotion of the user.
(Supplementary Note 18)The system according to Supplementary Note 1, wherein the instruction unit presents specific improvement points during instruction based on the user's brushing habits.
(Supplementary Note 19)The system according to Supplementary Note 1, wherein the instruction unit provides instruction content during instruction according to the condition of the user's oral cavity.
(Supplementary Note 20)The system according to Supplementary Note 1, wherein the instruction unit estimates the user's emotion and adjusts the level of detail of the instructions based on the estimated emotion of the user.
(Supplementary Note 21)The system according to Supplementary Note 1, wherein the instruction unit provides instruction content during instruction according to the user's age and gender.
(Supplementary Note 22)The system according to Supplementary Note 1, wherein the instruction unit proposes brushing methods during instruction according to the type of toothbrush used by the user.
(Supplementary Note 23)The system according to Supplementary Note 1, wherein the confirmation unit estimates the user's emotion and adjusts the display method of the confirmation result based on the estimated emotion of the user.
(Supplementary Note 24)The system according to Supplementary Note 1, wherein the confirmation unit adds a function to display areas of unbrushed areas during confirmation.
(Supplementary Note 25)The system according to Supplementary Note 1, wherein the confirmation unit adds a function to provide improvement points for the user's brushing method during confirmation.
(Supplementary Note 26)The system according to Supplementary Note 1, wherein the confirmation unit estimates the user's emotion and adjusts the frequency of confirmation based on the estimated emotion of the user.
(Supplementary Note 27)The system according to Supplementary Note 1, wherein the confirmation unit provides a confirmation method during confirmation according to the health condition of the user's oral cavity.
(Supplementary Note 28)The system according to Supplementary Note 1, wherein the confirmation unit customizes the confirmation result during confirmation based on the user's brushing history.

What is claimed is:

1. A system comprising:
circuitry configured to:
receive, via a communication interface communicatively coupled to a client terminal over a packet-switched network, image data captured by an imaging device of the client terminal;
analyze the image data using a convolutional neural network to generate analysis data indicating presence and location of at least one target region within the image data;
generate instruction data based on the analysis data, the instruction data indicating a remediation action directed to the at least one target region;
transmit the instruction data to the client terminal via the communication interface and the packet-switched network, the instruction data causing the client terminal to output a notification to a user;
receive, via the communication interface, subsequent image data captured by the imaging device after the user has performed the remediation action; and
analyze the subsequent image data to generate confirmation data indicating whether the at least one target region has been remediated.

2. The system according to claim 1, wherein the image data represents an interior of an oral cavity, and the at least one target region comprises at least one of tartar or an unbrushed area.

3. The system according to claim 1, wherein the circuitry is further configured to generate the analysis data by applying image processing comprising at least one of morphology processing, contour extraction, or heatmap generation to output of the convolutional neural network.

4. The system according to claim 1, wherein the circuitry is further configured to generate the analysis data comprising at least one of a binary mask indicating presence of the at least one target region, a probability map indicating likelihood of presence of the at least one target region, or a label identifying a location of the at least one target region.

5. The system according to claim 1, wherein the circuitry is further configured to generate the instruction data comprising at least one of a visual highlight of the at least one target region or a voice instruction indicating the remediation action.

6. The system according to claim 1, wherein the circuitry is further configured to:
estimate an emotion of the user based on at least one of facial expression data, voice data, or biometric data received from the client terminal; and
adjust a format of the instruction data based on the estimated emotion, such that when the estimated emotion indicates relaxation, the instruction data is generated in a detailed format, and when the estimated emotion indicates tension, the instruction data is generated in a concise format.

7. The system according to claim 6, wherein the circuitry is further configured to estimate the emotion by:
extracting facial features from a facial image using a convolutional neural network;
extracting acoustic features comprising mel-frequency cepstral coefficients from voice waveform data; and
integrating the facial features and the acoustic features to output an emotion class and an emotion score.

8. The system according to claim 1, wherein the circuitry is further configured to:
accumulate history data comprising the analysis data over a plurality of sessions; and
generate the instruction data based on the history data by identifying a pattern in the at least one target region across the plurality of sessions.

9. The system according to claim 8, wherein the circuitry is further configured to input the history data to a recurrent neural network to extract a trend in the at least one target region and generate personalized instruction data based on the extracted trend.

10. The system according to claim 1, wherein the circuitry is further configured to adjust timing of receiving the image data based on an estimated emotion of the user, such that the circuitry delays a request for the image data when the estimated emotion indicates tension.

11. The system according to claim 1, wherein the circuitry is further configured to transmit, to the client terminal, guide data indicating a target area for the imaging device to capture, the guide data causing the client terminal to display a visual indicator directing the user to position the imaging device toward the target area.

12. The system according to claim 1, wherein the circuitry is further configured to:
detect motion in the image data; and
apply a correction algorithm to reduce blur in the image data based on the detected motion.

13. The system according to claim 1, wherein the circuitry is further configured to select an imaging mode from a plurality of imaging modes based on a health condition indicated by prior analysis data, the plurality of imaging modes comprising a standard resolution mode and a high resolution mode.

14. The system according to claim 1, wherein the circuitry is further configured to generate the confirmation data comprising at least one of a comparison between the image data and the subsequent image data, or an improvement metric indicating a degree of remediation of the at least one target region.

15. The system according to claim 1, wherein the circuitry is further configured to:
generate the confirmation data indicating that the at least one target region has not been fully remediated; and
generate additional instruction data based on the confirmation data, the additional instruction data indicating a further remediation action.

16. The system according to claim 1, wherein the circuitry is further configured to generate product recommendation data based on the analysis data, the product recommendation data indicating at least one care product suitable for addressing the at least one target region.

17. The system according to claim 1, wherein the circuitry is further configured to customize the instruction data based on attribute information of the user comprising at least one of age, gender, or a type of tool used by the user for the remediation action.

18. A system comprising:
a communication interface configured to communicate with a client terminal via a packet-switched network;
a memory storing a data generation model obtained by deep learning on a neural network; and
circuitry configured to:
receive, via the communication interface, image data captured by an imaging device of the client terminal, the image data representing an interior of an oral cavity of a user;
analyze the image data using the data generation model to generate analysis data indicating presence and location of at least one of tartar or an unbrushed area within the oral cavity;
generate instruction data based on the analysis data, the instruction data indicating a brushing action directed to at least one of the tartar or the unbrushed area;
transmit the instruction data to the client terminal via the communication interface, the instruction data causing the client terminal to output a brushing instruction to the user;
receive, via the communication interface, subsequent image data captured by the imaging device after the user has performed the brushing action; and
analyze the subsequent image data using the data generation model to generate confirmation data indicating whether the at least one of the tartar or the unbrushed area has been addressed.

19. The system according to claim 18, wherein the circuitry is further configured to:
estimate an emotion of the user based on at least one of facial expression data or voice data received from the client terminal; and
adjust at least one of the instruction data or the confirmation data based on the estimated emotion.

20. A method performed by a system comprising circuitry, the method comprising:
receiving, via a communication interface communicatively coupled to a client terminal over a packet-switched network, image data captured by an imaging device of the client terminal;
analyzing, by the circuitry, the image data using a convolutional neural network to generate analysis data indicating presence and location of at least one target region within the image data;
generating, by the circuitry, instruction data based on the analysis data, the instruction data indicating a remediation action directed to the at least one target region;
transmitting the instruction data to the client terminal via the communication interface and the packet-switched network, the instruction data causing the client terminal to output a notification to a user;
receiving, via the communication interface, subsequent image data captured by the imaging device after the user has performed the remediation action; and
analyzing, by the circuitry, the subsequent image data to generate confirmation data indicating whether the at least one target region has been remediated.
